SEQUENCE LISTING (54 sequences)

SEQ ID NO: 1 - protein, 107 aa, Ophiophagus hannah
SPPGNWQKADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWL

SEQ ID NO: 2 - DNA, 321 bp, Ophiophagus hannah
tcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggctt

SEQ ID NO: 3 - protein, 127 aa, Ophiophagus hannah
MLLFTLCFFADQENGGKALASPPGNWQKADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWL

SEQ ID NO: 4 - DNA, 381 bp, Ophiophagus hannah
atgctcctgttcacactatgcttttttgctgaccaggaaaatggtggaaaggctctggcttcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggctt

SEQ ID NO: 5 - protein, 170 aa, Ophiophagus hannah
SPPGNWQKADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWLRRLETDSDKLQKGSGKIIVFLDYDEGKVIFDLDGEVTTIQANFNGEEVVPFYYIGARVSLANL

SEQ ID NO: 6 - DNA, 507 bp, Ophiophagus hannah
tcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttccactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctt

SEQ ID NO: 7 - protein, 190 aa, Ophiophagus hannah
MLLFTLCFFADQENGGKALASPPGNWQKADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWLRRLETDSDKLQKGSGKIIVFLDYDEGKVIFDLDGEVTTIQANFNGEEVVPFYYIGARVSLANL

SEQ ID NO: 8 - DNA, 567 bp, Ophiophagus hannah
atgctcctgttcacactatgcttttttgctgaccaggaaaatggtggaaaggctctggcttcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttccactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctt

SEQ ID NO: 9 - DNA, 1584 bp, Ophiophagus hannah
gatcatttgatccagagaagacacagtctcctggactcattattgaaaagaagactcccaatcggatcctgcacaatagttttatctccctagggaaaaaaaatattcaagagaaaatttgaataaaggaagctagagtttgtaatggtcatgtccctttctgctggcttccaatttagtttgaacttccaacaaacaaagaaagtcctgcggaagctcacaggagtctcttgcatgctcctgttcacactatgcttttttgctgaccaggaaaatggtggaaaggctctggcttcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttccactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctttaagaggttacaattcttcattaaaacaggggacttctctctacagtctttgcaatgcctgttcaagcatttaataatctctggctttgggagaataatcatgcacaggtagtgctcgacttacaacagttggtttagtggctgtttgaagttacaacggcactggaaaatataacttatgaccattttcacacttatgaccgttgcagcattcccatagtcacgtggtcaaaaaacagtcacttgacaactgccttatatttatgacggttgccgtgtcccagcatcatgtgatcagcttttgttaccttctgagaagcaaaatcagtggggaagccagattcacttaacaaccatgttactaacttaacaactacagtgattcacttagcaactgtggctagaaaggtcataaaatcaggcaaaactcacttaacaactgtttcacttaacaacagaatttggggctcaattgtgctcataagtcgaggactacctgcatcatgtgtccttctccatccccaaactgccaacagcatttaaattccagatgtataagggttgttgccatacactttacactcctttgtttaaacaatattgcatccttttccccttagacccataaaagtttcttgaagggaattacggcaaggccatattgaaggttgtcccagtaatataccaaatctgtttaatcaattggacaaaaggaatcatatctgtactactaataaagtctcactgttagtaaaaaaaaaaaaaaaaaaaaaaaaaaaaaaa

SEQ ID NO: 10 - DNA, 1843 bp, Ophiophagus hannah
tcacctcctgggaattggcagaagggtaagagatggcttaaataagagtgtccttctccaggatcagtcgggtcaggtgaacaataatgccagacaatttcattgcaattaagtttggatttgctagatatttcctggcctcctttgtatgatccaacaagagtactcctaacatcagagtctcttgttgagatagaaatcaaataaataaatctattgtggatggaagggagaacagagccatgtgaaatttaaccaacaaacattggctttcccagcttgcacctcactatagcttcagctgtgtgggcattttcccttattctgacactgtataagctgtcaggagtcatcttggtgagatgggtggcgtagaagtctaataataaataaatagagtcaaatgtggccttgagatgtataaggtctgctaggctttgtcatctcagagcagtgtttatcaacctctgctagttggagtgcagaatagttttttaaatgggctagcaggatacaagaaacagtgcaacagtgaccccataactgtaggcagcccaagtctccttttttgaatggtcagaagcctttttgacaatgatccttgaaaactaaccgcctgtaatcaattacttgcaaaaaaaaagggtagtggtaaaaatgaaccatattggagaactgaggatcacagcatttgttgcgtcatagcaatagcaatggcatttagacttatacaccactttacaccactctctaagtggttacagagtcagcatgctgcccccaacaatctgggtcctcattttaccaacctcagaaggatggaaggctcagtcaaccttgagcctggtgagaatcgaacttcaggctgtgagcagagtttgcctacaataccccattctacccactgtgtcaccacagctcatatcacatatacatgagggtgtgcctaaacatacattattttacattacatactgtacacacattattcttttgttcataaatgtgactatttgggagagaagtgtgagggattttgttttctagaaagccacagtgcacctcttacctaccactccacttttgacttgttttaggagagcagagttttacaacatcattgaagaagcagtcagcctttattgcactatatatctttacataaagccctattaaaaagccttgtgtttatgcatcatataaatatgtagaagaatgcagacatgggaaatgaagccgggatattttgtggcctttcttgcccctttaatttgaggtgggtgggtaataacattttcagtgatgttgctctcctagagtttggtttggcttctgatgttctatctttctctctgcagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttcggcgactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctt

SEQ ID NO: 11 - DNA, 106 bp, Artificial Sequence
  Oligonucleotide primer
ggaattcgtcgacggatccatggctagcccgccgggtaactggcagaaagcggacgtcaccttcgatagcaacaccgcgttcgaaagcctggtggtgagcccggac

SEQ ID NO: 12 - DNA, 117 bp, Artificial Sequence
  oligonucleotide primer
tccccccgggctgcctaggacgcacgggctcgaggagaagcgttccgggctatccggcacacctttcggcacaccaacgttttccacggtttttttgtccgggctcaccaccaggct

SEQ ID NO: 13 - DNA, 107 bp, Artificial Sequence
  oligonucleotide primer
tccccccgggtttccgttccggaaaacacttcttcgaggtgaaatacggtacccagcgtgaatgggcggtggggctagcgggtaaaagcgtgaagcgtaagggttac

SEQ ID NO: 14 - DNA, 95 bp, Artificial Sequence
  oligonucleotide primer
gactagtaagcttgcggccgcctacagccaccacagacctttctgccagatacgttcttccgcaccagccttaagtaacccttacgcttcacgct

SEQ ID NO: 15 - DNA, 21 bp, Artificial Sequence
  oligonucleotide primer
ggnaaytggcaraargcngay

SEQ ID NO: 16 - DNA, 21 bp, Artificial Sequence
  oligonucleotide primer
ccaccanarnccyttytgcca

SEQ ID NO: 17 - DNA, 29 bp, Artificial Sequence
  oligonucleotide primer
cttcccagctaacccaacagcccattccc

SEQ ID NO: 18 - DNA, 30 bp, Artificial Sequence
  oligonucleotide primer
gatcatttgatccagagaagacacagtctc

SEQ ID NO: 19 - DNA, 36 bp, Artificial Sequence
  oligonucleotide primer
gtcgacggatccatgtcacctcctgggaattggcag

SEQ ID NO: 20 - DNA, 38 bp, Artificial Sequence
  oligonucleotide primer
aagcttgcggccgcttaaagatttgcgagtgaaacacg

SEQ ID NO: 21 - DNA, 18 bp, Artificial Sequence
  oligonucleotide primer
aagatttgcgagtgaaac

SEQ ID NO: 22 - DNA, 18 bp, Artificial Sequence
  oligonucleotide primer
aagatttgcgagtgaaac

SEQ ID NO: 23 - protein, 99 aa, Ophiophagus hannah
ADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWL

SEQ ID NO: 24 - protein, 100 aa, Naja kaouthia
ADVTFDSNTAFESLVVSPDKKTVENVGVSQVAPDNPERFDGSPCVLGSPGFRSGKHFFEVKYGTQREWAVGLAGKSVKRKGYLRLVPEERIWQKGLWWLG

SEQ ID NO: 25 - protein, 138 aa, Artificial Sequence
  synthetic peptide
VDVTLDPDTAHPYLSLSEDRKSVRYGDLKQSLPDNPERFDHYPCVLGSQGFSGKHYFEVEVFTGGDKGHWRVGWATKSVPRGGFRLLGEDKGSWGYDGDGGKKYHNSEFPEYGLPFQEPGDVIGCFLDLEAGTISFYK

SEQ ID NO: 26 - protein, 136 aa, Artificial Sequence
  Synthetic peptide
VDVTLDPDTAYPSLILSDNLRQVRYSYLQQDLPDNPERFNLFPCVLGSPCFIAGRHYWEVEVGDKAKWTIGVCEDSVCRKGGVTSAPQNGFWAVSLWYGKEYWALTSPMTALPLRTPLQRVGIFLDYDAGEVSFYN

SEQ ID NO: 27 - protein, 135 aa, Bos taurus
VDVTLDPDTAHPHLFLYEDSKSVRLEDSRQKLPEKPERFDSWPCVMGREAFTSGRHYWEVEVGDRTDWAIGVCRENVMKKGFDPMTPENGFWAVELYGNGYWALTPLRTPLPLAGPPRRVGVFLDYESGDIFFYN

SEQ ID NO: 28 - protein, 139 aa, Synanceia horrida
CDLTFDRNTINNWISLSDNDTFAASEHGKRQNYPKHPERFVSFNQVLCNEGLMGKHYWEVEWNGYIDVGIAYISIPRKEIDFASAFGYNTYSWVLSYNPKIGYIERHKKREYNVRAPNPGFKRLGLFLDWRYGSISFYA

SEQ ID NO: 29 - protein, 140 aa, Homo sapiens
RTPTLDPDTMHARLRLSADRLTVRCGLLGSLGPVPVLRFDALWQVLARDCFATGRHYWEVDVQEAGAGWWVGAAYASLRRRGASAAARLGCNRQSWCLKRYDLEYWAFHDGQRSACGPATTSTGSASSWTTRPASSPSTT

SEQ ID NO: 30 - protein, 146 aa, Homo sapiens
VDVMLNPGSATSNVAISVDQRQVKTVRTCTFKNSNPCDFSAFGVFGCQYFSSGKYYWEVDVSGKIAWILGVHSKISSLNKRKSSGFAFDPSVNYSKVYSRYRPQYGYWVIGLQNTCEYNAFEDSSSSDPKVLTLFMAVLPVVLGFS

SEQ ID NO: 31 - DNA, 370 bp, Artificial Sequence
  Synthetic gene for ohanin (sense strand)
ggaattcgtcgacggatccatggctagcccgccgggtaactggcagaaagcggacgtcaccttcgatagcaacaccgcgttcgaaagcctggtggtgagcccggacaaaaaaaccgtggaaaacgttggtgtgccgaaaggtgtgccggatagcccggaacgcttctcctcgagcccgtgcgtcctaggcagcccgggtttccgttccggaaaacacttcttcgaggtgaaatacggtacccagcgtgaatgggcggtggggctagcgggtaaaagcgtgaagcgtaagggttacttaaggctggtgccggaagaacgtatctggcagaaaggtctgtggtggctgtaggcggccgcaagcttactagtc

SEQ ID NO: 32 - DNA, 370 bp, Artificial Sequence
  Synthetic gene for ohanin (antisense)
gactagtaagcttgcggccgcctacagccaccacagacctttctgccagatacgttcttccggcaccagccttaagtaacccttacgcttcacgcttttacccgctagccccaccgcccattcacgctgggtaccgtatttcacctcgaagaagtgttttccggaacggaaacccgggctgcctaggacgcacgggctcgaggagaagcgttccgggctatccggcacacctttcggcacaccaacgttttccacggtttttttgtccgggctcaccaccaggctttcgaacgcggtgttgctatcgaaggtgacgtccgctttctgccagttacccggcgggctagccatggatccgtcgacgaattcc

SEQ ID NO: 33 - protein, 115 aa, Ophiophagus hannah
EFVDGSMASPPGNWQKADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWL

SEQ ID NO: 34 - DNA, 1558 bp, Ophiophagus hannah
gatcatttgatccagagaagacacagtctcctggactcattattgaaaagaagactcccaatcggatcctgcacaatagttttatctccctagggaaaaaaaaatattcaagagaaaatttgaataaaggaagctagagtttgtaatggtcatgtccctttctgctggcttccaatttagtttgaacttccaacaaacaaagaaagtcctgcggaagctcacaggagtctcttgcatgctcctgttcacactatgcttttttgctgaccaggaaaatggtggaaaggctctggcttcacctcctgggaattggcagaaagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttcggcgactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctttaagaggttacaattcttcattaaaacaggggacttctctctacagtctttgcaatgcctgttcaagcatttaataatctctggctttgggagaataatcatgcacaggtagtgctcgacttacaacagttggtttagtggctgtttgaagttacaacggcactggaaaatataacttatgaccattttcacacttatgaccgttgcagcattcccatagtcacgtggtcaaaaaacagtcacttgacaactgccttatatttatgacggttgccgtgtcccagcatcatgtgatcagcttttgttaccttctgagaagcaaaatcagtggggaagccagattcacttaacaaccatgttactaacttaacaactacagtgattcacttagcaactgtggctagaaaggtcataaaatcaggcaaaactcacttaacaactgtttcacttaacaacagaatttggggctcaattgtgctcataagtcgaggactacctgcatcatgtgtccttctccatccccaaactgccaacagcatttaaattccagatgtataagggttgttgccatacactttacactcctttgtttaaacaatattgcatccttttccccttagacccataaaagtttcttgaagggaattacggcaaggccatattgaaggttgtcccagtaatataccaaatctgtttaatcaattggacaaaaggaatcatatctgtactactaataaagtctcactgttagta

SEQ ID NO: 35 - protein, 190 aa, Ophiophagus hannah; identical to SEQ ID NO: 7

SEQ ID NO: 36 - protein, 162 aa, Ophiophagus hannah
ADVTFDSNTAFESLVVSPDKKTVENVGVPKGVPDSPERFSSSPCVLGSPGFRSGKHFFEVKYGTQREWAVGLAGKSVKRKGYLRLVPEERIWQKGLWWLRRLETDSDKLQKGSGKIIVFLDYDEGKVIFDLDGEVTTIQANFNGEEVVPFYYIGARVSLANL

SEQ ID NO: 37 - protein, 100 aa, Naja kaouthia; identical to SEQ ID NO: 24

SEQ ID NO: 38 - protein, 168 aa, Artificial Sequence
  Synthetic peptide
ADVTLDPETAHPNLVLSEDRKSVKFVETRLRDLPDTPRRFTFYPCVLATEGFTSGRHYWEVEVGDKTHWAVGVCRDSVSRKGELTPLPETGYWRVRLWNGDKYAATTTPFTPLHIKVKPKRVGIFLDYEAGTLSFYNVTDRSHIYTFTDTFTEKLWPLFYPGIRAGRK

SEQ ID NO: 39 - protein, 166 aa, Artificial Sequence
  Synthetic peptide
VDVTLDPDTAHPHLFLYEDSKSVRLEDSRQILPDRPERFDSWPCVLGRETFTSGRHYWEVEVGDRTDWAIGVCRENVVKKGFDPMTPDNGFWAVELYGNGYWALTPLRTSLRLAGPPRRVGVFLDYDAGDISFYNMSNGSLIFPYTSISFSGPLRPFFCLWSCGKK

SEQ ID NO: 40 - protein, 168 aa, Artificial Sequence
  Synthetic peptide
VDVTLDPDTAHPYLSLSEDRKSVRYGDLKQSLPDNPERFDHYPCVLGSQGFSGRHYFEVEVFTGGDKGHWRVGWATKSVPRGGFRLLGEDKGSWGYDGDGGKKYHNSEFPEYGLPFQEPGDVIGCFLDLEAGTISFYKNGKYLGLAFFDVTFSGPLYPAVSLGNGGSV

SEQ ID NO: 41 - protein, 168 aa, Synanceia horrida
CELTLDPETAHQVLTLSEGNKKAVSGNTKSPTDHLEKFSHFQQVMCTKGLSGRHYWELEWSGYVGAGVTYKGIGRKTSTSDSSLGKNEKSWLFEYSTKSGYQQIHNSKKTRVTVSSTGFKLLGVYLDWPAGTLSFYMVNKAWVTHLHTFHTKFNEAVYPAFLIGDAQQ

SEQ ID NO: 42 - protein, 170 aa, Synanceia horrida
CDLTFDRNTINNWISLSDNDTFAASEHGKRQNYPKHPERFVSFNQVLCNEGLMGKHYWEVEWNGYIDVGIAYISIPRKEIDFASAFGYNTYSWVLSYNPKIGYIERHKKREYNVRAPNPGFKRLGLFLDWRYGSISFYAVSSDEVHHLHTFKTKFTEPVYPAFSIGPAGN

SEQ ID NO: 43 - protein, 165 aa, Cavia porcellus
ANVTLDPYTAHPALILSEERRVSMGEKPQDLPRSQMRFESLPCVLGKQSFSSERHFWEVKVNSCTGWDLGICRSNVMRKERTYIKPEDGFWAIRFYNHEYWALTSPKTQLTLKKPPGKVCIFLDFEDQRISFYNMTDNSHIYTFSQGAFYGSLRPFFRLWSKDSG

SEQ ID NO: 44 - protein, 180 aa, Mus musculus
LDQLLDMPAAGLAVQLRHAWNPEDRSLNVFVKDDDRLTFHRHPVAQSTDGIRGKVGHARGLHAWQIHWPARQRGTHAVVGVATARAPLHSVGYTALVGSDSESWGWDLGRSRLYHDGKNRPGVAYPAFLGPDEAFALPDSLLVVLDMDEGTLSFIVDGQYLGVAFRGLKGKKLYPVVSAV

SEQ ID NO: 45 - DNA, 7086 bp, Ophiophagus hannah
gatcatttgatccagagaagacacagtctcctggactcattattgaaaagaaggtaagatcttacaacgcagctttcttccagaaatgctgattacttacccttgcttgtgaatcctgtaatagcttgggctgaagatagaatgtggaaaggatcttttattttccaagggctcaaaaagctttttgcggcagttgacacacaaagttctgcattttatcagaactcaaatttttgaagtaaaatgggaatatttctgccttttacctcgttaactcctagaaaaacatattgaagaaaaaggctagactagagcattattcactatccatcagctcaattatttgtttgtgaaatgaaccttgtagagatcatcagtgattaaatgcttcatcagctgttggtgtatacatgccagatcattttcaggcataatcctgcacaataggtttttttttaaaaaactttattaaattttattacattgaaaaacaacaaaacaaaaaacataaaaaacaaaaaaaacataagacataaaaacattgaaaaaaaacattaaaagtatatcactcaatgacaactacttgcggtgataattacctaacaatcatatgtgcatgactaacaaatgttaacttgtgcataaatatatttttattctatctatatccaatatttactgattacctatctttattctgtctccgctctatccaactgtaccattgttcctatactttgtaaaatattgaatcttctttgtgttttaaactcaacgatagtttatccacttcggcacagctcataattttttgtaagaccaattcatctgctggcatatgtggctgtttccagcatagagcaaaagcaatcctagcagcagtcaatatatgtattaataaatatcttgtgctttttacatttttttaaagaattcccaatagaaagagttctggttctagatctaactgttcattagtaatttcctgaagccatttgtggattttgagccaatatttgtgagcttctgggcaagaccaccacatatggtagtatgttccctcttttttttttacacttccaacacatgagactcattttagaatacattttcgctaatctcgaagggggtaagtgccatctgtaaaacattttgtatgtgttttctttgtaagttactgattttgttaatttgtggtttctattccatatagactcccaatcggatcctgcacaatagttttatctccctagggaaaaaaaaatattcaagagaaaatttgaataaaggtagacctcagcttatgaccatttgtttgaagttatgctggcccccaaaaagagtttcttatgactcatctttgaagttatgtcacactacccctgtgttcatgtgactggaatgtgggcatttggcaaccagcttgcatttaagaggttgccgtgtcctgcaatcatgtgactgtgatttgcaatcttcgcagccagcttctgatcaagcaaagtgaattggggaagccagatttgcttaatgacagtgtgattcatttaatgaccatgtaatttgcttaacaactgaggaagtttacttaagagccgtggtggcacagtggttagaatgcagtactgcaggctactcctgccagcagttcggttctcaccagactcaaggttgactcagccttccatccttccaaggtcggtaaaatgaggatccagactgttgggggcaatatgctagctctgtaaactgcttagagagggctgtaaagcagtataaagcagtatataagtctaagtgctattgctattaatgaccacagtaaaattgatcataaaattgggtccagtcacatgatcatttacttagcaactgcatttgcttagtgactgaaattccggtcccaattaaattgagaactatctgtagactcatcaacaagttgagatattggaattgagaacttgggactggtagtcacaagttgcggcttagattttgagtgattaaaataaactcttgagaaatgccatttcccccacccccccacccccatttttcttctattggcatattcattattttcctctaaaaaagtggaataatgaggaaatgctaattagaatttataaaaccacagagataaacaataatgctgattttttaaagtttaaactgattactttgttttacacttaaaactgagttctgtaggaaacagacttccagcgaagaaaacccaatccaaagagaacctactggaattcaatttctcaccatcctgaactcaaaactataaagtgacaaaagccttccaagacctatcaaactgctgaaactgcactatgcacataccactgaaaacaggctgtatatggtactaaaaaaacacgcacactactttatgatgctcatgaaagcattgcatggcttcagcggtttgtgggctcaaggaaggagaatgtcatctgccattttgtgtgagatcatgttaccaggcagccatttgctgttttaagtactgtaacaagtcggccatctgccattttgaaacaggcaagcccattaatatctaggggttgactttaaaagtcagatgttcttaaatgggggacttcctgcactaaacagataaaattctagcagaagacttcgtaaactgttacacaaggtcttttggaacctgttgtgtggaattgtgcaacactttgcatgtccttctatgaaaacctgtgctgcaatccttcctgaaatgttggctgagtgttcaaccaactcactgcaaaatagaaggacagttttggatatcaatccttttgtagttcacaaaagtcttcaaaatattatttattggtcactcacctgagaatgaactgtgtcagatgactcacagaaaaacagaataaaaaaatgatgacatggtataaaacagaatgaaaaaacgatgacattatatgtcatgctctggattcacttgaactaagttttaaatccatttaatcatttactcattttctctattccttctgtaggaagctagagtttgtaatggtcatgtccctttctgctggcttccaatttagtttgaacttccaacaaacaaagaaagtcctgcggaagctcacaggtaaagaaatatatatatatatatcatgattgtggtttgcttttgctctttcaacactttcagctctcttttgtgcaaaaatatcttggcaactccagtgtcccctttgatcatatggtgcacacttttccccagtctgatcaaacactccccttccattaggtatgtttgctgccttctagttatatattttttaaaagatgggataaaaatataaagataatgccttcatatgttgcaaaacattttctttctgctttggtcatcactgagctatagcttataactatatgctttttgtcatattttgttcatgtactggtaaagtcttgatcactatagattgctgggaaagacagatttatcgtagaagggaaaaggaaatatatcccattgtgcatttatcagccagtcagccagtcttctccaaatatctcctccaaacaaatgaaacagaatactatatttggctaaccagggaacaaaagttagaacattcccaagacctgcaagacattataggcataaacaatagcactcagacctatataccacttcacagtgctttacagccctctccaagagtttacagtgtcagcatattgccccccaacaatcagagtcctcattttaccgacctcagaaggatggaaggctgagtcaaccatgagctggtgagaattgaactgctggcagtcggcagaattaacctcaatgctgcattctaaccactgtgccaccacaactcttgacttatgcccataattgaacccacaatttcagttgtaagtcatgacagtcattacacatcatatgattacacccgatcttattaccttttttgtagcaatcataaggcttacgtagtggttgtaaaaagaacactatggttgttaagcaaatgccacagttgttaaacaaacccattgtttgttatggggagttttttgccggaagctggaagtagatgctggtttttgttaaaaatgccataaatggtggtcatgtgaccacgggacatggcaaaaaggtcatcaaaatgcactcatggccatgggcagagttgtggatctgggtggatgtatgtgtgtggatccagatattggtacttcaaatccaggtagtaagtagcttttttggaatccattgtaagttgaggactacctgtacaggacatgtgacagcaagtgttaaattatgcccagtttttctgtaaaccaaactttaccatctataagacagtataaacaatgccttacctattcttgcctttgtgttctgtccatgtttaggagtctcttgcatgctcctgttcacactatgcttttttgctgaccaggaaaatggtggaaaggctctggcttcacctcctgggaattggcagaaaggtaagagatggcttaaataagagtgtccttctccaggatcagtcgggtcaggtgaacaataatgccagacaattacattgcaattaagtttggatttgctagatatttcctggcctcctttgtatgatccaacatgagtactcctaacatcagagtctcttgttgagatagaaatcaaataaataaatctattgtggatggaagggagaacagagtcatgtgaaatttaaccaacaaacattggctttcccagcttgcacctcactatagcttcagctgtgtggggattttcctttattctgacactgtataagctgtcaggagtcatcttggtgagatgggtggcgtagaagtctaataataaataaatagagtcaaatgtggccttgagatgtataaggtctgctaggctttgtcatctcagagcagtgtttatcaacctctgctagttggagtgcagaatagttttttaaatgggctagcaggatacaagaaacagtgcaacagtgaccccataactgtaggcagcccaagtctccttttttgaatggtcagaagcctttttgacaatgattcttgaaaactaaccgcctgtaatcaattacttgcaaaaaaaaagggtagtggtaaaaatgaaccatattggagaactgaggatcacagcatttgttgcgtcatagcaatagcaatggcatttagacttatacaccactttacaccactctctaagtggttacagagtcagcatgctgcccccaacaatctgggtcctcattttaccaacctcagaaggatggaaggctcagtcaaccttgagcctggtgagaatcgaactccaggctgtgagcagagtttgcctacaataccccattctacccactgtgtcaccacagctcatatcacatatacatgagggtgtgcctaaacatacattattttacattacatactgtacacacattattcttttgttcataaatgtgactatttgggagagaagtgtgagggattttgttttctagaaagccacagtgcacctcttacctatcactccacttttgacttgttttaggagagcagagttttacaacatcattgaagaagcagtcagcctttattgcactatatatctttacataaagccctattaaaaagccttgtgtttatgcatcatataaatatgtagaagaatgcagacatgggaaatgaagccgggatattttgtggcctttcttgcccctttaatttgaggtgggtgggtaataacattttcagtgatgttgctctcctagagtttggtttggcttctgatgttctatctttctctctgcagctgatgtgacgtttgactcaaacacagcatttgaatcactggttgtgtcaccagacaagaagactgtggaaaatgtgggtgtccccaagggtgtgccagacagtccagagagattcagtagcagcccctgtgtgctggggtctcctggattccgatcagggaagcatttctttgaagtgaagtatgggacacaaagggaatgggctgttgggttagctgggaagtctgtgaagagaaaagggtacctcagacttgtccccgaagaacggatttggcaaaagggcctctggtggcttcggcgactggaaacggattctgacaagcttcaaaaaggctctggaaagattattgtctttctggattacgatgagggaaaagtgatttttgacctggatggtgaagtcactaccatccaggccaatttcaatggggaggaagttgtaccgttttactatataggggcacgtgtttcactcgcaaatctttaagaggttacaattcttcattaaaacaggggacttctctctacagtctttgcaatgcctgttcaagcatttaataatctctggctttgggagaataatcatgcacaggtagtgctcgacttacaacagttggtttagtggctgtttgaagttacaacggcactggaaaatataacttatgaccattttcacacttatgaccgttgcagcattcccatagtcacgtggtcaaaaaacagtcacttgacaactgccttatatttatgacggttgccgtgtcccagcatcatgtgatcagcttttgttaccttctgagaagcaaaatcagtggggaagccagattcacttaacaaccatgttactaacttaacaactacagtgattcacttagcaactgtggctagaaaggtcataaaatcaggcaaaactcacttaacaactgtttcacttaacaacagaatttggggctcaattgtgctcataagtcgaggactacctgcatcatgtgtccttctccatccccaaactgccaacagcatttaaattccagatgtataagggttgttgccatacactttacactcctttgtttaaacaatattgcatccttttccccttagacccataaaagtttcttgaagggaattacggcaaggccatattgaaggttgtcccagtaatataccaaatctgtttaatcaattggacaaaaggaatcatatctgtactactaataaagtctcactgttagta

SEQ ID NO: 46 - protein, 190 aa, Ophiophagus hannah; identical to SEQ ID NO: 7

SEQ ID NO: 47 - DNA, 10 bp, Artificial Sequence
  synthetic DNA
agaaggtaag

SEQ ID NO: 48 - DNA, 10 bp, Artificial Sequence
  Synthetic DNA
taaaggtaga

SEQ ID NO: 49 - DNA, 10 bp, Artificial Sequence
  Synthetic oligonucleotide
cacaggtaaa

SEQ ID NO: 50 - DNA, 10 bp, Artificial Sequence
  Synthetic oligonucleotide
gaaaggtaag

SEQ ID NO: 51 - DNA, 10 bp, Artificial Sequence
  Synthetic oligonucleotide
tatagactcc

SEQ ID NO: 52 - DNA, 10 bp, Artificial Sequence
  Synthetic oligonucleotide
tgtaggaagc

SEQ ID NO: 53 - DNA, 10 bp, Artificial Sequence
  Synthetic oligonucleotide
tttaggagtc

SEQ ID NO: 54 - DNA, 10 bp, Artificial Sequence
  Synthetic oligonucleotide
tgcagctgat

(19) United States
(12) Patent Application Publication
(10) Pub. No.: US 2009/0285825 A1
(43) Pub. Date: Nov. 19, 2009

(54) NOVEL SNAKE TOXIN

(75) Inventors: Manjunatha Ramachandra Kini, Kent Vale (SG); Yuh Fen Fung, Malacca (MY); Prakash Pallathadka Kumar, Singapore (SG); Peter Tsun Hon Wong, Singapore (SG); Chung Pin Teo, Singapore (SG)

Correspondence Address: KNOBBE MARTENS OLSON & BEAR LLP, 2040 MAIN STREET, FOURTEENTH FLOOR, IRVINE, CA, 92614, US

(73) Assignee: National University of Singapore, Singapore (SG)

(21) Appl. No.: 11/721,286

(22) PCT Filed: Dec. 22, 2005
(86) PCT No.: PCT/SG2005/000429

§ 371 Date: Feb. 16, 2009

Related U.S. Application Data
(60) Provisional application No. 60/639,345, filed on Dec. 22, 2004.

(51) Int. Cl. C07K 14/435; C12N 15/12; C12N 15/70; C12N 1/21; C12P 21/02; C40B 50/06; A61K 39/38; C40B 50/04; A61K 39/395; C07K 16/18; A61K 38/17; A61K 31/7088; A61K 48/00

(52) U.S. Cl. 424/141.1; 530/350; 536/23.1; 536/23.4; 435/320.1; 435/252.33; 435/69.1; 435/69.7; 506/25; 530/387.9; 514/12; 514/44 R; 424/93.2

(57) ABSTRACT

This invention is in the field of snake venom and the invention provides a novel snake toxin protein and nucleic acids encoding the same. Also, provided are various uses and compositions based on the discovery of the novel snake toxin.

All documents cited herein are incorporated by reference in their entirety.

### TECHNICAL FIELD

This invention is in the field of snake venom and the invention provides a novel snake toxin protein and nucleic acids encoding the same. Also, provided are various methods and compositions based on the discovery of the novel snake toxin.

### BACKGROUND ART

Snake venoms are complex mixtures of bioactive compounds, including enzymatic and non-enzymatic proteins, as well as low molecular weight components including peptides, lipids, nucleotides, carbohydrates and amines (1-2). Venom proteins generally serve in a number of adaptive roles: immobilizing, paralyzing, killing and digesting prey (3). Hence, snake venoms serve both offensive and defensive purposes (4). Over the past 40 years, a plethora of toxin proteins have been isolated and characterized from venoms of snakes (5). These toxin proteins, however, belong to a very small number of structural superfamilies of proteins (6). The members of a superfamily share similar molecular scaffold, but, at times, exhibit distinct biological functions.

The major enzyme groups found in snake venoms include phospholipases, serine proteinases, metalloproteinases, phosphodiesterases, acetylcholinesterase, L-amino acid oxidases and nucleases (2, 7). Generally, enzymes in the venom have molecular weight ranging from 13,000 Da to 150,000 Da. Most of these are hydrolases and possess a digestive role. On the other hand, over 1000 non-enzymatic venom proteins have been characterized and they are grouped into three-finger toxins, serine proteinase inhibitors, lectins, sarafatoxins, nerve growth factors, atrial natriuretic peptides, bradykinin-potentiating peptides, helveprins/CRISP proteins, disintegrins and waprins (6-9). Non-enzymatic polypeptide toxins have molecular weight around 1,000 Da to 25,000 Da and are rich in disulphide bonds. Therefore, they are robust and are relatively stable once isolated. The low molecular weight compounds have molecular weight less than 1,500 Da. They are less active biologically and are presumed to be enzyme cofactors (2).

### SUMMARY OF THE INVENTION

We have identified, purified and determined the complete amino acid sequence of a novel protein, ohanin from Ophiophagus hannah (king cobra) venom. It is a small protein containing 107 amino acid residues with a molecular mass of 11951.47±0.67 Da as assessed by ESI-mass spectrometry. It does not show similarity to any known families of snake venom proteins and hence is the first member of a new family of snake venom proteins. It shows similarity to PRY and SPRY domains (a domain with unknown function in Ryanodine receptors and Dictyostelium discoideum) and B30.2 domain. It was non-lethal up to the dose of 10 mg/kg when given i.p. At doses of 1 mg/kg and 10 mg/kg, mice injected with ohanin seemed to be quiet with sluggish movements. Therefore, its effects on the motor activity of mice were examined using an infrared ray sensor. Ohanin produced statistically significant and dose-dependent hypolocomotion in mice. In hot plate assay, it showed dose-dependent hyperalgesic effect.

The ability of the protein to elicit a response at greatly reduced doses when injected intracerebroventricularly as compared to intraperitoneal administration in both the locomotion and hot plate experiments strongly suggests that ohanin acts on the central nervous system. It was 6,500-fold more potent when administered i.c.v. (intracerebroventricularly) than i.p. (intraperitoneally). Since the natural abundance of the protein in the venom is low (˜1 mg/g), a synthetic gene was constructed and expressed. The recombinant protein, which was obtained in the insoluble fraction in E. coli, was purified under denaturing condition and was refolded. Recombinant ohanin is structurally and functionally similar to native protein as determined by circular dichroism and hot plate assay, suggesting that it will be useful in future structure-function relationship studies.

In order to further characterize this novel protein, we have determined the sequence of the cDNA coding for ohanin. Interestingly, its cDNA does not show significant sequence similarity to any known sequences in the GenBank data base, including those of B30.2-like domain-containing protein families. It was found that there are two mRNA subtypes differing in their 5′-untranslated regions. The full-length cDNA sequence is 1558 bp in length, excluding the poly-A tail. It has a 20-residue signal peptide, followed by 107 residues of mature ohanin and 63 residues of pro-peptide segment at the C-terminal region of the mature protein. Ohanin has a complete SPRY domain that spans across to the propeptide region. This new protein family was named vespryns (venom PRY-SPRY domain-containing proteins). However, unlike all other B30.2-like domain-containing protein families that have relatively long N-terminal segments, ohanin has only eight residues preceding its PRY-SPRY domains.

Pro-ohanin was expressed in E. coli as a soluble fusion protein with hexahistidine tagged at the N-terminal. Similar to ohanin, recombinant pro-ohanin was also assessed for its biological functions in mice. It should be noted that analyses from both the locomotor activity and hot plate assay strongly indicate that pro-ohanin did not exhibit similar pharmacological actions in intraperitoneally-administered mice as compared to the mature ohanin. But pro-ohanin shows shows potent hypolocomotion and hyperalgesia effects when injected directly into the mice ventricles. The large size and/or conformation changes of the proprotein may have inhibited pro-ohanin from crossing the blood-brain barrier and subsequently preventing its interaction with molecular target(s) at the central nervous system. Interestingly, although the presence of propeptide segment inhibits the ability of ohanin to cross the blood-brain barrier, it enhances the pharmacological action at the central nervous system. It should be noted that pro-ohanin is 35-fold more potent than ohanin when the injection is given via i.c.v. route. Furthermore, pro-ohanin at 0.3 μg/kg is able to block ˜90% of the locomotor activity of the experimental mice.

We have also studied the genomic organization of ohanin gene. Southern hybridization indicates that ohanin is encoded by a single gene in the king cobra genome. Genomic DNA sequence analysis shows that ohanin gene is 7086 bp; and contains five exons and four introns. The two types of mRNAs observed are generated through alternative splicing in snake venom genes. Similar genomic organization among ohanin and other B30.2-like domain-containing proteins indicates that the B30.2-like domains of these proteins may have evolved from a common ancestral and adapted to function in the venom glands.

This is the first snake venom protein reported so far which induces hypolocomotion and hyperalgesia in experimental mice. It is envisaged that Ohanin will be useful in the development of prototypes of new pharmaceutical agents or as research tools.

Accordingly, a first aspect of the invention includes:

(a) a protein comprising the mature sequence of ohanin as set forth in SEQ ID NO. 1 (amino acid sequence of ohanin excluding its signal peptide);
(b) a protein comprising the mature sequence of ohanin and its signal peptide as set forth in SEQ ID NO. 3 (amino acid sequence of ohanin and its signal peptide);
(c) a protein comprising the amino acid sequence of pro-ohanin as set forth in SEQ ID NO. 5 (amino acid sequence of pro-ohanin excluding the signal peptide); and
(d) a protein comprising the amino acid sequence of pro-ohanin and its signal peptide as set forth in SEQ ID NO. 7 (amino acid sequence of pro-ohanin including the signal peptide).

Typically the proteins of the first aspect of the invention include natural biological variants, such as allelic variants. Also included are functional equivalents which contain single or multiple amino-acid substitution(s), addition(s), insertion(s) and/or deletion(s) and/or substitutions of chemically-modified amino acids, wherein “functional equivalent” denotes a protein that: (i) retains the ability of the protein to induce at least one of hypolocomotion or hyperalgesia; or (ii) which has an antigenic determinant in common with the protein. Also included are active fragments wherein “active fragment” denotes a truncated protein that: (i) retains the ability of the protein to induce at least one of hypolocomotion or hyperalgesia; or (ii) which has an antigenic determinant in common with the protein. Also included are fusion proteins wherein the protein is fused to a peptide or other protein, such as a label, which may be, for instance, bioactive, radioactive, enzymatic or fluorescent, or an antibody.

For the avoidance of doubt, the first aspect of the invention includes: functional equivalents of the natural biological variants; active fragments of the natural biological variants and functional equivalents; and fusion proteins comprising the natural biological variants, functional equivalents and active fragments.

A second aspect of the invention provides a nucleic acid molecule which encodes a protein according to the first aspect of the invention.

A third aspect of the invention provides a vector, such as an expression vector, that contains a nucleic acid molecule of the second aspect of the invention.

A fourth aspect of the invention provides a host cell transformed with a vector of the third aspect of the invention.

A fifth aspect of the invention provides a method of producing a protein according to the first aspect of the invention, the method comprising culturing a host cell according to the fourth aspect of the invention under conditions suitable for the expression of the protein of the first aspect of the invention. The method of the fifth aspect of the invention may further comprise purifying the protein.

A sixth aspect of the invention provides a method of producing a protein according to the first aspect of the invention the method comprising the chemical synthesis of the protein by, for example, solid-phase peptide synthesis or combinatorial chemistry.

A seventh aspect of the invention provides a method of making an antibody which is capable of binding to a protein of the first aspect of the invention.

An eighth aspect of the invention provides an antibody which is capable of binding to a protein of the first aspect of the invention.

A ninth aspect of the invention provides a method of producing an antivenom against a protein according to the first aspect of the invention wherein the method comprises immunizing an animal with a protein according to the first aspect of the invention and harvesting antibodies from the animal for use as the antivenom.

A tenth aspect of the invention provides an antivenom effective against a protein of the first aspect of the invention. Preferably, the antivenom is produced in accordance with the ninth aspect of the invention.

An eleventh aspect of the invention provides a method for identifying a modulator (e.g. an agonist or antagonist) compound of a polypeptide of the first aspect of the invention.

A twelfth aspect of the invention provides a pharmaceutical composition comprising a protein of the first aspect of the invention, a nucleic acid molecule of the second aspect of the invention, a vector of the third aspect of the invention, a host cell of the fourth aspect of the invention, an antibody of the eighth aspect of the invention, an antivenom of the tenth aspect of the invention, or a modulator (e.g. an agonist or antagonist) identified by the eleventh aspect of the invention.

A thirteenth aspect of the invention provides a protein of the first aspect of the invention, a nucleic acid molecule of the second aspect of the invention, a vector of the third aspect of the invention, a host cell of the fourth aspect of the invention, an antibody of the eighth aspect of the invention, an antivenom of the tenth aspect of the invention, or a modulator (e.g. an agonist or antagonist) identified by the eleventh aspect of the invention for use in medicine.

A fourteenth aspect of the invention provides for the use of a protein of the first aspect of the invention, a nucleic acid molecule of the second aspect of the invention, a vector of the third aspect of the invention or a host cell of the fourth aspect of the invention in the manufacture of a medicament for use as a sedative.

A fifteenth aspect of the invention provides for the use of a protein of the first aspect of the invention, a nucleic acid molecule of the second aspect of the invention, a vector of the third aspect of the invention or a host cell of the fourth aspect of the invention in the manufacture of a medicament for use in the treatment of a neurological or muscular disease.

A sixteenth aspect of the invention provides a method of sedating an animal comprising administering to the animal a protein of the first aspect of the invention, a nucleic acid molecule of the second aspect of the invention, a vector of the third aspect of the invention, a host cell of the fourth aspect of the invention or a pharmaceutical composition of the twelfth aspect of the invention.

A seventeenth aspect of the invention provides a method of treating a patient with a neurological or muscular disease comprising administering to the patient a protein of the first aspect of the invention, a nucleic acid molecule of the second aspect of the invention, a vector of the third aspect of the invention, a host cell of the fourth aspect or a pharmaceutical composition of the twelfth aspect of the invention of the invention.

An eighteenth aspect of the invention provides a defensive composition comprising a protein of the first aspect of the invention.

### BRIEF DESCRIPTION OF DRAWINGS

FIG. 1. Search for novel proteins in king cobra venom. Crude venom (60 μg) was loaded on to a RP-Jupiter C18 analytical column attached to the Perkin-Elmer Sciex API300 LC/MS/MS mass spectrometry. The bound proteins were eluted using a linear gradient of 80% ACN in 0.1% TFA (v/v) at a flow rate of 50 μl/min. The peak containing protein of interest is indicated with arrow.

FIG. 1A. Masses of peptides and proteins detected by LC/MS from king cobra venom.

FIG. 2. Isolation and purification of the novel protein. (A) Gel filtration of king cobra venom. Crude venom (200 mg) was loaded onto a Superdex 30 column (Hiload 16/60). The column was pre-equilibrated with 50 mM Tris-HCl (pH 7.4). Proteins were eluted at a flow rate of 1 ml/min in the same buffer. The horizontal solid bar (peak 1b) indicates the fraction containing the protein of interest. (B) RP-HPLC of peak 1b from gel filtration. Jupiter C18 semi-preparative column was equilibrated with 0.1% (v/v) TFA. The protein of interest was eluted from the column at a flow rate of 2 ml/min with a gradient of 38 to 40% B (80% ACN in 0.1% TFA). The arrow indicates the peak corresponding to the protein of interest. (C) ESI/MS of the novel protein. The protein has a molecular mass of 11951.47±0.67 Da as indicated by Biospec Reconstruct spectrum.

FIG. 3. Purification of peptide digests. Lys-C endopeptidase-digested peptides (A); Tryptic peptides (B); and Formic acid-digested peptides (C).

FIG. 3D. Theoretical and experimentally determined masses of peptides of ohanin.

FIG. 4. Amino acid sequence of ohanin. The protein sequence was determined by Edman degradation. Bold

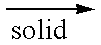

arrow, N-terminal sequence of native and pyridylethylated ohanin;

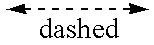

arrows, Lys-C peptides;

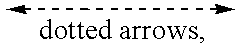

tryptic peptides; and

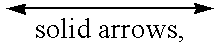

formic acid-digested peptides.

FIG. 5. Sequence alignment of B30.2-like domain of ohanin with other B30.2-like domain-containing proteins. Proteins containing B30.2-like domain are: ohanin (sp: P83234), Thaicobrin (sp: P82885), PRY-SPRY domain (conserved sequences of PRY-SPRY domains were obtained from the CDD database), RFP (Ring Finger Protein, gb: J03407), BTN (Butyrophilin, sp: P18892), Alpha SNTX (α-subunit of Stonustoxin, gb: U36237), KIAA0129 (gb: D50919) and Staf50 (gb: X82200). Identical and conserved residues are shaded black and grey, respectively. Three conserved LDP, WEVE and LDYE motifs are boxed. The numbers in parentheses represent the percentage of similarity between the B30.2-like domains of the proteins. The substitutions among the following groups of amino acid residues are considered as conserved changes: Y, F, and W; S and T; V, L, I and M; H, R and K; D and E; N and Q; A and G.

FIG. 6. Effect of ohanin on locomotor activity of mice. Cumulative (A) and time course (B) of locomotor activity following i.p. injection (n=8 to 9) of ohanin. Cumulative (C) and time course (D) of locomotor activity following i.c.v. injection (n=7 to 9). The dose-dependent inhibition of locomotor activity is over 6,500-fold more potent with i.c.v. injection of ohanin. Data represent mean counts of locomotor activity±S.E.M.; one-way ANOVA was used in (A) and (C) and two-way ANOVA in (B) and (D); post-hoc analysis by Bonferroni's test: a, P<0.05; b, P<0.01 and c, P<0.001.

FIG. 7. Hyperalgesic effect of native and recombinant ohanin. Latency time in a hot plate assay at 55° C. following i.p. injection (n=8) (A); and i.c.v. injection (n=8 to 16) (B) of native ohanin. (C) Latency time (n=8 to 16) following i.c.v. injection of recombinant ohanin. Both the native and recombinant ohanin showed a dose-dependent hyperalgesic effect for low and intermediate doses when administered i.c.v. Data represent mean latency time±S.E.M.; one-way ANOVA followed by Bonferroni's test: a, P<0.05 and b, P<0.01.

FIG. 8. Design, construction and cloning of a synthetic gene for ohanin. (A) Schematic representation of the expression construct. Thrombin and CNBr cleavage sites are indicated as Tb and CNBr, respectively. The DNA fragment between the two arrows was inserted into vectorM at the BamHI and NotI sites. (B) Full-length sequence of the synthetic gene. Amino acid sequence is shown below the DNA sequence. Ten unique restriction sites (AatlI, BstBI, AclI, XhoI, AvrII, XmaI, BspEI, KpnI, NheI and AflII) are in bold. (C) Strategy for construction of the synthetic gene. Two pairs of oligonucleotides, ranging from 96 bp to 117 bp in length, were used to assemble the two fragments (P1 and P2). These two fragments were then ligated via XmaI site to generate the entire gene (see Experimental Procedures for details). Agarose gel (1.5%, w/v) electrophoresis of the two fragments (D) and the synthetic gene (E).

FIG. 9. SDS-PAGE analysis of recombinant ohanin. Samples were resolved in 15% polyacrylamide gel and stained with Coomassie Brilliant Blue-R250. (A) Expression and solubility of recombinant ohanin in E. coli. Lane M, Prestained broad range standards, Lanes 1-5, total protein sample from bacterial culture after 0, 10, 12, 14 and 16 h, respectively, after IPTG induction; Lanes 6 and 7, protein samples from supernatant and pellet after sonication. (B) Purification and refolding of fusion protein. Lane M, Precision plus prestained dual-color standard; Lanes 1 and 2, total protein samples from first and second rounds of sonification; Lane 3, elution of fusion protein from the affinity column under denaturing conditions; Lane 4, empty lane; Lane 5, refolded fusion protein. (C) Cleavage of fusion protein by CNBr. Lane M, Precision plus prestained dual-color standard; Lanes 1 and 2, fusion protein before and after cleavage. The fusion peptide (˜2 kDa) is too small to be resolved by 15% polyacrylamide gel. Bands labeled 1, 2 and 3 are expressed fusion protein, lysozyme and recombinant ohanin, respectively.

FIG. 10. Purification of recombinant ohanin. (A) RP-HPLC of recombinant ohanin after thrombin cleavage. The arrow indicates the fraction containing the recombinant protein. (B) ESI/MS of recombinant ohanin. The recombinant protein has a molecular mass of 12226.91±0.89 Da as indicated by Biospec Reconstruct spectrum.

FIG. 11. CD spectra of the native and recombinant ohanin. (A) CD spectra were recorded using a 2 mm-path-length cuvette. Measurement was made in MilliQ water. The concentration used was 12.5 μM. CD spectra of native and recombinant ohanin are shown in bold and thin lines, respectively. (B) Structural contents of native and recombinant ohanin.

FIG. 12. Cloning and sequencing of ohanin cDNA. (A) 5′-RACE amplification. Partial coding region of ohanin together with its 5′-UTR were obtained from the 5′-RACE amplification using GSP2 and UPM. (B) 3′-RACE amplification. The 3′-RACE amplification which yielded the full-length cDNA of 1558 bp exclusive of poly-A tail was obtained using GSP1 and UPM. (C) Nucleotide sequence and deduced amino acid sequence of ohanin (gb:AY351433). Nucleotides are presented in the 5′- to 3′-orientation. Deduced amino acid sequence by reverse-translation of the longest open reading frame is shown: the putative signal peptide is underlined; ohanin is marked in bold; dibasic cleavage site is boxed and pro-peptide segment is marked in italic. The stop codon is indicated by an asterisks and the polyadenylation signal, AATAAA, is underlined twice. The missing stretch of nucleotides in type II cDNA is shaded black.

FIG. 13. Sequence alignment of B30.2-like domains. The domains are from Pro-ohanin (gb: AY351433), Thaicobrin (sp: P82885), RFP (Ring finger protein, gb: NM 172016), BTN (Butyrophilin, gb: NP 038511), PRY-SPRY domains (conserved sequences of PRY-SPRY domains were provided by CDD database), Beta subunit of SNTX (β-subunit Stonustoxin, gb: Q91453), Alpha subunit of SNTX (α-subunit Stonustoxin, gb: □98989), Enterophilin (gb: AF126833) and SPRY domain-containing SOCS box protein 4 (Suppressors of cytokine signaling, gb: NP 660116). Identical and conserved residues are shaded black and grey. The Arg-Arg dibasic cleavage site of pro-ohanin is indicated in bold and propeptide segment is marked in italics. Three conserved LDP, WEVE and LDYE motifs are boxed. Gaps (−) were introduced for optimal alignment and maximum homology for the sequences. The arrows indicate the boundary of PRY and SPRY domains. The numbers in parentheses represent the percentage of similarity between the B30.2-like domain of pro-ohanin with other B302-like domain-containing proteins. The substitutions among the following groups of amino acid residues are considered as conserved changes: Y, F, and W; S and T; V, L, I and M; H, R and K; D, E, N and Q; A and G.

FIG. 14. Schematic representation of proteins possessing B30.2-like domain. B 30.2-like domains are shaded black and the unidentified domains are dotted.

FIG. 15. Expression construct of pro-ohanin. (A) Agarose gel (1.5%, w/v) electrophoresis of the nucleotide sequence corresponding to pro-ohanin amplified from 19K1 and 19K2 primers. (B) Schematic representation of the expression construct. Thrombin and CNBr cleavage sites are indicated as Tb and CNBr, respectively. The cDNA fragment of 530 bp corresponding to pro-ohanin was inserted between the two arrows at BamHI and NotI sites.

FIG. 16. SDS-PAGE analysis of recombinant pro-ohanin. Samples were resolved in 15% SDS-PAGE gels and stained with Coomassie Brilliant Blue-R250. (A) Expression of recombinant pro-ohanin in E. coli. Lane M, Precision plus prestained dual-color standard; Lanes 1, Total protein sample from bacterial culture before induction; Lane 2, After IPTG induction. (B) Purification of fusion protein. Lane M, Precision plus prestained dual-color standard; Lanes 1 and 2, Elution of fusion protein from the affinity column under non-denaturing conditions. (C) Cleavage of fusion protein by thrombin. Lane M, Precision plus prestained dual-color standard; Lanes 1 and 2, Fusion protein before and after cleavage. Bands labeled 1 and 2 are the expressed fusion protein and recombinant pro-ohanin, respectively. The fusion peptide (˜2 kDa) is too small to be resolved by 15% SDS-PAGE gel.

FIG. 17. Purification of recombinant pro-ohanin using RP-HPLC. The horizontal bar indicates the peak corresponding to recombinant pro-ohanin.

FIG. 18. CD spectra comparison between ohanin and pro-ohanin. (A) CD spectra were recorded for 12.5 μM of proteins in MilliQ water using a 2-mm path-length cuvette. Bold line, ohanin; thin line, pro-ohanin. (B) Secondary structural contents of ohanin and pro-ohanin.

FIG. 19. Hypolocomotion effect of ohanin and pro-ohanin. Cumulative locomotor activity following intraperitoneal injection (n=8 to 9) of ohanin (A) and pro-ohanin (B). Cumulative locomotor activity following i.c.v. injection (n=6 to 9) of ohanin (C) and pro-ohanin (D). Data represent mean counts of locomotor activity±S.E.M.; one-way ANOVA was used; post-hoc analysis by Bonferroni's test: a, P<0.05; b, P<0.01 and c, P<0.001.

FIG. 20. Hyperalgesic effect of ohanin and pro-ohanin. Latency time in hot plate assay at 55° C. following intraperitoneal injection (n=8) (A) and (B); i.c.v. injection (n=8 to 16) (C) and (D). (A) and (C), latency time obtained from ohanin-injected mice; (B) and (D), latency time obtained from pro-ohanin-injected mice. When administered via i.c.v. route, ohanin shows a dose-dependent hyperalgesic effect for low and intermediate doses; whereas pro-ohanin has a relatively shorter latency time for all the doses. Data represent mean latency time±S.E.M.; one-way ANOVA followed by Bonferroni's test: a, P<0.05 and b, P<0.01.

FIG. 21. Genomic Southern blot of ohanin. Genomic DNA of king cobra (10 μg each lane) was digested with EcoRI, HindIII, BamHI or NdeI enzymes. Southern hybridization shows the presence of one single band in all four digests. Thus ohanin is encoded by a single gene in the king cobra genome. The migration position of λHindIII marker is indicated.

FIG. 22. Ohanin gene sequence. Using both the genomic DNA PCR and ‘genome walking’ strategies, the full-length genomic sequence of 7086 bp was obtained. Exon-intron boundaries were determined based on cDNA and genomic sequences. Exons are shaded grey and indicated by upper case letters while introns are indicated by lower case letters. The missing exon in type II cDNA is shaded black. The three ATGs are indicated in bold; the putative signal peptide is underlined; dibasic processing site is boxed; propeptide segment is marked in italics, the stop codon is indicated by an asterisk and the polyadenylation signal, AATAAA, is underlined twice.

FIG. 23. Genomic organization of ohanin. Ohanin gene comprises of five exons and four introns. Exons 1 to 5 have the sizes of 53, 76, 95, 96 and 1238 bp, respectively. The introns are 1160, 1743, 1292 to 1333 bp, respectively. In the case of alternative splicing, the whole exon 2 is excluded producing a shorter transcript of 1482 bp. The complete cDNA was named type I, while the shorter cDNA corresponding to the alternative splicing (missing exon 2) was named type II cDNA.

FIG. 23A. The exon-intron boundaries of ohanin gene.

FIG. 24. Strategy for in vitro binding studies of His-pro-ohanin.

FIG. 25. Immunofluorescent slides showing fluorescence in in vitro binding assays of His-pro-ohanin to hippocampus and cerebellum regions of the brain in comparison to non-fluorescent control experiments without pre-incubation with His-pro-ohanin.

FIG. 26. Schematic showing a competition binding control assay for assessing binding specificity of His-pro-ohanin

FIG. 27. Immunofluorescent slides showing binding specificity of His-pro-ohanin compared to controls

FIG. 28. Strategy for in vivo binding study of His-ohanin and His-pro-ohanin.

FIG. 29. Immunofluorescent slides showing the dose-dependent binding of ohanin and pro-ohanin in the hippocampus (A) and cerebellum (B) regions of the brain.

FIG. 30. Immunofluorescent slides showing the ability of ohanin and pro-ohanin in crossing the blood-brain barrier.

### DETAILED DESCRIPTION

A first aspect of the invention includes:
(a) a protein comprising the mature sequence of ohanin as set forth in SEQ ID NO. 1 (amino acid sequence of ohanin excluding its signal peptide);
(b) a protein comprising the mature sequence of ohanin and its signal peptide as set forth in SEQ ID NO. 3 (amino acid sequence of ohanin and its signal peptide);
(c) a protein comprising the amino acid sequence of pro-ohanin as set forth in SEQ ID NO. 5 (amino acid sequence of pro-ohanin excluding the signal peptide) and
(d) a protein comprising the amino acid sequence of pro-ohanin and its signal peptide as set forth in SEQ ID NO. 7 (amino acid sequence of pro-ohanin including the signal peptide).

The proteins of the first aspect of the invention include natural biological variants, such as allelic variants. Also included are functional equivalents which contain single or multiple amino-acid substitution(s), addition(s), insertion(s) and/or deletion(s) and/or substitutions of chemically-modified amino acids, wherein “functional equivalent” denotes a protein that: (i) retains the ability of the protein to induce at least one of hypolocomotion or hyperalgesia; or (ii) which has an antigenic determinant in common with the protein. Also included are active fragments wherein “active fragment” denotes a truncated protein that: (i) retains the ability of the protein to induce at least one of hypolocomotion or hyperalgesia; or (ii) which has an antigenic determinant in common with the protein. Also included are fusion proteins wherein the protein of the invention is fused to a peptide or other protein, such as a label, which may be, for instance, bioactive, radioactive, enzymatic or fluorescent, or an antibody.

For the avoidance of doubt, the first aspect of the invention includes: functional equivalents of the natural biological variants; active fragments of the natural biological variants and functional equivalents; and fusion proteins comprising the natural biological variants, functional equivalents and active fragments.

The terms “polypeptide” and “protein” are used interchangeably and refer to any polymer of amino acids (dipeptide or greater) linked through peptide bonds or modified peptide bonds, whether produced naturally or synthetically. Polypeptides of less than about 10-20 amino acid residues are commonly referred to as “peptides.”

The proteins of the invention may also comprise non-peptidic components, such as carbohydrate groups. Carbohydrates and other non-peptidic substituents may be added to a protein by the cell in which the protein is produced, and will vary with the type of cell. Proteins are defined herein, in terms of their amino acid backbone structures; substituents such as carbohydrate groups are generally not specified, but may be present nonetheless. However, given that native ohanin is believed to lack post-translational modifications it is preferred that the proteins of the first aspect of the invention do not contain any post-translational modifications such as glycosylation or disulfide bridges. Of course, however, where the proteins of the first aspect of the invention are expressed as pro-proteins then the proteins may undergo processing to the mature form. Thus, it is preferred that the mature proteins of the first aspect of the invention or the proteins of the first aspect of the invention when processed into their mature form do not contain any post-translational modifications such as glycosylation or disulfide bridges.

The term “comprising” and grammatical variants thereof as used herein means “including” or “consisting”. Thus, for example, a composition “comprising” X may consist exclusively of X or may include one or more additional components. Similarly, a polypeptide or nucleic acid molecule comprising a given sequence may consist exclusively of the given sequence or may include one or more additional components.

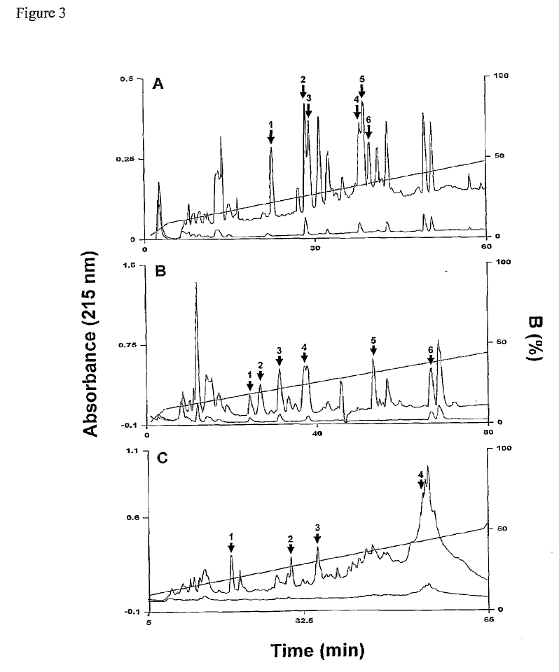

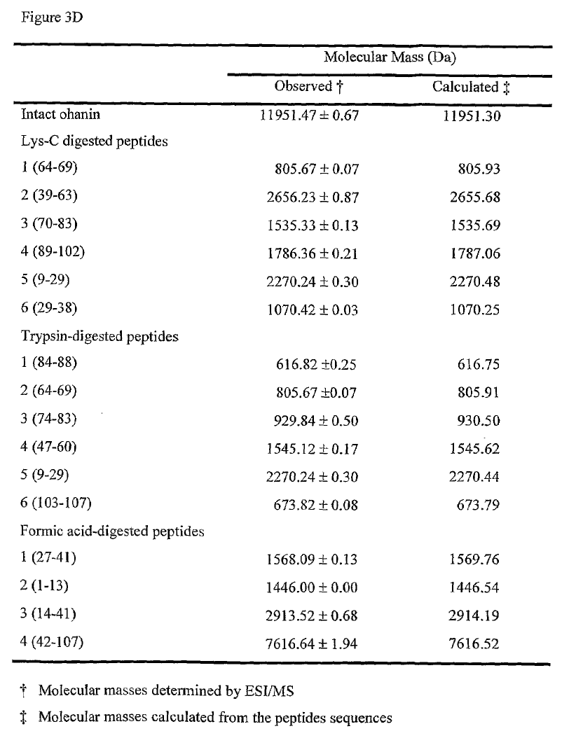

In one embodiment of the first aspect of the invention there is provided a polypeptide which comprises the mature ohanin amino acid sequence as set forth in SEQ ID NO. 1. SEQ ID NO. 1 consists of 107 amino acids and is shown in FIG. 3. In one embodiment there is provided a polypeptide which consists of the mature ohanin amino acid sequence as set forth in SEQ ID NO. 1.

In another embodiment of the first aspect of the invention there is provided a polypeptide which comprises the amino acid sequence as set forth in SEQ ID NO. 3. SEQ ID NO. 3 consists of the mature sequence of ohanin (i.e. the 107 amino acids of SEQ ID NO.1) preceded by its 20 amino acid signal peptide sequence. The signal peptide sequence is the underlined sequence in FIG. 9. In one embodiment there is provided a polypeptide which consists of the amino acid sequence as set forth in SEQ ID NO. 3.

In another embodiment of the first aspect of the invention there is provided a polypeptide which comprises the amino acid sequence as set forth in SEQ ID NO. 5. SEQ ID NO. 5 is the pro-ohanin sequence, i.e. the 107 amino acids of SEQ ID NO.1 and the 63 amino acid C-terminal pro-sequence. In one embodiment there is provided a polypeptide which consists of the amino acid sequence as set forth in SEQ ID NO. 5.

In another embodiment of the first aspect of the invention there is provided a polypeptide which comprises the amino acid sequence as set forth in SEQ ID NO. 7. SEQ ID NO. 7 is the pro-ohanin sequence preceded by the 20 amino acid signal peptide sequence, i.e. SEQ ID NO. 7 consists of the 107 amino acid sequence of FIG. 3 and the 63 amino acid C-terminal pro-sequence and the signal sequence. SEQ ID NO. 7 is shown in FIG. 9. In one embodiment there is provided a polypeptide which consists of the amino acid sequence as set forth in SEQ ID NO. 7.

In one embodiment of the invention there is provided a natural biological variant of a protein of the invention in particular of a protein as set forth in SEQ ID NO. 1, 3, 5 or 7. Natural biological variants include allelic variants within the species from which the polypeptides are derived. Such variants may include polypeptides in which one or more of the amino acid residues are substituted with a conserved or non-conserved amino acid residue (preferably a conserved amino acid residue). Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Particularly preferred are natural variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. “Mutant” polypeptides also include polypeptides in which one or more of the amino acid residues include a substituent group.

A further embodiment of the first aspect of the invention provides functional equivalents of the proteins of the invention (in particular of SEQ ID NO. 1, 3, 5 or 7 and natural biological variants thereof) that contain single or multiple amino-acid substitution(s), addition(s), insertion(s) and/or deletion(s) from the wild type protein sequence and/or substitutions of chemically-modified amino acids, wherein “functional equivalent” denotes a protein that (i) retains the ability of the protein to induce at least one of hypolocomotion or hyperalgesia; or (ii) which has an antigenic determinant in common with the protein.

It will of course be appreciated that where the ability of a protein to induce hypolocomotion or hyperalgesia is referred to and the protein is an inactive pro-protein (at least when administered intraperitoneally) the reference to its ability to induce hypolocomotion or hyperalgesia is a reference to its ability to induce the same when processed into its mature (active) form or when administered i.c.v.

Methods for determining the ability of a protein to induce hypolocomotion or hyperalgesia are known in the art. Also, methods for determining the ability of a protein to induce hypolocomotion or hyperalgesia are described in the Examples section. The methods described in the Examples section may suitably be used to determine the ability of a protein to induce hypolocomotion or hyperalgesia.

Preferably, a protein of the first aspect of the invention retains at least 30%, 40%, 50%, 60%, 70%, 80%, or 90% of the potency of ohanin to induce hypolocomotion. The ability of a protein to induce hypolocomotion vis-à-vis the ability of ohanin (SEQ ID NO.1) to induce hypolocomotion may be assessed by comparing the effect of the proteins on locomotion using the method described in the Examples section herein. The effect of the proteins may be compared when they are both administered i.p. or i.c.v. Comparisons may be performed when the proteins are administered at dosages of, for example, 0.1 mg/kg, 1 mg/kg and 10 mg/kg i.p. Comparisons may also be performed when the proteins are administered at dosages of, for example, 0.3 μg/kg, 1 μg/kg or 10 μg/kg administered i.c.v.

In one embodiment, a protein of the first aspect of the invention retains at least 30%, 40%, 50%, 60%, 70%, 80%, or 90% of the potency of ohanin to induce hyperalgesia although as stated elsewhere it may, in certain embodiments, be advantageous for the protein to have reduced or absent ability to induce hyperalgesia. The ability of a protein to induce hyperalgesia vis-à-vis the ability of ohanin (SEQ ID NO.1) to induce hyperalgesia may be assessed by comparing the effect of the proteins on nociception of thermal pain using the method described in the Examples section herein. The effect of the proteins may be compared when they are both administered i.p. or i.c.v. Comparisons may be performed when the proteins are administered at dosages of, for example, 0.1 mg/kg, 1 mg/kg and 10 mg/kg i.p. Comparisons may also be performed when the proteins are administered at dosages of, for example, 0.3 μg/kg, leg/kg or 10 μg/kg administered i.c.v.

A functionally-equivalent polypeptide according to this aspect of the invention may be a polypeptide that is homologous to a polypeptide of the invention. Preferably, a functionally-equivalent polypeptide according to this aspect of the invention may be a polypeptide that is homologous to a polypeptide whose sequence is explicitly recited herein such as SEQ ID NO. 1, 3, 5 or 7.

Two polypeptides are said to be “homologous” if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences.

Methods of measuring protein homology are well known in the art and it will be understood by those of skill in the art that in the present context, homology is calculated on the basis of amino acid identity (sometimes referred to as “hard homology”). For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403 Software for performing BLAST analyses is publicly available through the National Center for Biotechnology Information on the world wide web through the internet at, for example, “www.ncbi.nlm nih.gov/”. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the both strands. The BLAST algorithm performs a statistical analysis of the similarity between two sequences; see e.g., Karlin and Altschul (1993) Proc. Nad. Acad. Sci. USA 90: 5873 One measure of similarity provided by the BLAST algorithm is the smallest sum probability (P(N)), which provides an indication of the probability by which a match between two nucleotide or amino acid sequences substituted for each other.

Typically, greater than 60% homology between two proteins is considered to be an indication of functional equivalence, provided that either the biological activity (the ability to induce at least one of hyperalgesia and hypolocomotion) of the protein is retained or the protein possesses an antigenic determinant in common with the protein. Preferably, a functionally equivalent polypeptide according to this aspect of the invention exhibits a degree of sequence identity with the polypeptide, or with a fragment thereof, of greater than 60%. More preferred polypeptides have degrees of homology of greater than 70%, 80%, 90%, 95%, 98% or 99%, respectively.

Functionally-equivalent polypeptides according to the invention are therefore intended to include mutants (such as mutants containing amino acid substitutions, insertions or deletions). Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. “Mutant” polypeptides also include polypeptides in which one or more of the amino acid residues include a substituent group.

Functional equivalents with improved function may also be designed through the systematic or directed mutation of specific residues in the protein sequence. One improvement that may be desired may be the reduction or abolition of the polypeptide's hyperalgesic function. This may be desirable where the polypeptide is being employed for its ability to induce hypolocomotion/sedation or where it is being administered to animals to raise antibodies.

Active fragments of the invention should comprise at least n consecutive amino acids from a polypeptide of the invention. Suitably, the active fragment should comprise at least n consecutive amino acids from a polypeptide according to SEQ ID NO. 1, 3, 5 or 7. n preferably is 7 or more (for example, 8, 10, 12, 14, 16, 18, 20, 50, 100, 150 or more). Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. Additionally, several fragments may be comprised within a single larger polypeptide.

In one embodiment of the first aspect of the invention there is provided a functional equivalent or an active fragment which has an antigenic determinant in common with a protein of the invention. Preferably, the antigenic determinant is shared with a polypeptide which consists of the amino acid sequence as set forth in SEQ ID NO. 1, 3, 5 or 7 or a natural variant thereof.

“Antigenic determinant” refers to that fragment of a molecule (i.e., an epitope) that makes contact with a particular antibody. “Antigenic determinants” or epitopes usually consist of chemically active surface groupings of molecules such as amino acids or sugar side chains and have specific three dimensional structural characteristics as well as specific charge characteristics.

Preferably, the functional equivalent or active fragment has an antigenic determinant in common with the amino acid sequence as set forth in SEQ ID NO. 1.

It is known in the art that relatively short synthetic peptides that can mimic antigenic determinants of a protein can be used to stimulate the production of antibodies against the protein (see, for example, Sutcliffe et al., Science 219:660 (1983)). Antigenic epitope-bearing peptides and polypeptides can contain at least four to ten amino acids, at least ten to fifteen amino acids, or about 15 to about 30 amino acids of SEQ ID NO:1. Such epitope-bearing peptides and polypeptides can be produced by fragmenting SEQ ID NO:1, or by chemical peptide synthesis, as described herein. Moreover, antigenic determinants can be selected by phage display of random peptide libraries (see, for example, Lane and Stephen, Curr. Opin. Immunol. 5:268 (1993), and Cortese et al., Curr. Gpin. Biotechnol. 7.616 (1996)). Standard methods for identifying antigenic determinants and producing antibodies from small peptides that comprise an antigenic determinant are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies Production, Engineering, and Clinical Application, Ritter and Ladyman (eds.), pages 6084 (Cambridge University Press 1995), and Coligan et al. (eds.), Current Protocols in Immunology, pages 9 1-9 5 and pages 9 1-9 11 (John Wiley & Sons 1997).

Such polypeptides possessing an antigenic determinant can be used to generate ligands, such as polyclonal or monoclonal antibodies, that are immunospecific for the polypeptides of the invention. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

In one embodiment of the first aspect of the invention there is provided a fusion protein comprising a protein of the invention fused to a peptide or other protein, such as a label, which may be, for instance, bioactive, radioactive, enzymatic or fluorescent, or an antibody.

For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Fusion proteins may also be useful to screen peptide libraries for inhibitors of the activity of the polypeptides of the invention. It may be useful to express a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein. By a “heterologous protein”, we include a protein which, in nature, is not found in association with a polypeptide of the invention.

In a preferred embodiment of the first aspect of the invention there is provided a protein which comprises the amino acid sequence as set forth in SEQ ID NO. 1, 3, 5 or 7. Preferably, the protein consists of the amino acid sequence as set forth in SEQ ID NO. 1, 3, 5 or 7.

A second aspect of the invention provides a nucleic acid molecule which encodes a protein according to the first aspect of the invention.

In one embodiment of the second aspect of the invention the nucleic acid molecule may comprise a nucleic acid sequence encoding an amino acid sequence as set forth in SEQ ID NO. 1, 3, 5 or 7.

In one embodiment of the second aspect of the invention the nucleic acid molecule may consist of a nucleic acid sequence encoding a protein which consists of the amino acid sequence as set forth in SEQ ID NO. 1, 3, 5 or 7.

In one embodiment the nucleic acid molecule may comprise the sequence which is set forth in SEQ ID NO. 2 which encodes the amino acid sequence set forth in SEQ ID NO. 1. In one embodiment the nucleic acid molecule may consist of the sequence which is set forth in SEQ ID NO. 2.

In another embodiment the nucleic acid molecule may comprise the sequence which is set forth in SEQ ID NO. 4 which encodes the amino acid sequence set forth in SEQ ID NO. 3. In one embodiment the nucleic acid molecule may consist of the sequence which is set forth in SEQ ID NO. 4.

In another embodiment the nucleic acid molecule may comprise the sequence which is set forth in SEQ ID NO. 6 which encodes the amino acid sequence set forth in SEQ ID NO. 5. In one embodiment the nucleic acid molecule may consist of the sequence which is set forth in SEQ ID NO. 6.

In one embodiment the nucleic acid molecule may comprise the sequence which is set forth in SEQ ID NO. 8 which encodes the amino acid sequence set forth in SEQ ID NO. 7. In one embodiment the nucleic acid molecule may consist of the sequence which is set forth in SEQ ID NO. 8.

In another embodiment the nucleic acid molecule may comprise the nucleic acid sequence as set forth in SEQ ID NO. 9 (the full-length cDNA sequence of ohanin/pro-ohanin including the signal peptide sequence). In one embodiment the nucleic acid molecule may consist of the nucleic acid sequence as set forth in SEQ ID NO. 9

In another embodiment the nucleic acid molecule may comprise the nucleic acid sequence as set forth in SEQ ID NO. 10 (the genomic DNA sequence of ohanin/pro-ohanin excluding the signal peptide sequence). In one embodiment the nucleic acid molecule may consist of the nucleic acid sequence as set forth in SEQ ID NO. 10.

It will be appreciated by those skilled in the art that as a result of the degeneracy of the genetic code, a multitude of nucleic acid molecules encoding the proteins of the first aspect of the invention, some bearing minimal homology to the polynucleotide sequences of any known and naturally occurring gene, may be produced. Thus, the invention contemplates each and every possible variation of polynucleotide sequence that could be made by selecting combinations based on possible codon choices.

Moreover, those skilled in the art will appreciate that codons may be selected to increase the rate at which expression of the peptide occurs in a particular prokaryotic or eukaryotic host in accordance with the frequency with which particular codons are utilized by the host.

Nucleic acids of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

The term “nucleic acid molecule” also includes analogues of DNA and RNA, such as those containing modified backbones.

In one embodiment of the second aspect of the invention there is provided nucleic acid molecules which are homologous with a nucleic acid molecule which encodes a protein which comprises (and optionally consists) of the amino acid sequence as set forth in SEQ ID NO. 1, 3, 5 or 7.

In one embodiment, there is provided nucleic acids which are homologous with a nucleic acid sequence as set forth in SEQ ID NO. 2, 4, 6, 8, 9 or 10.

In a specific embodiment, two DNA sequences are “homologous” when at least about 70%, and most preferably at least about 80%, 85%, 90%, 95%, 97%, 98% or 99% of the nucleotides match over the defined length of the DNA sequences, as determined by sequence comparison algorithms.

The degree of homology between two nucleic acid sequences may be determined by means of computer programs known in the art such as GAP provided in the GCG program package (Program Manual for the Wisconsin Package, Version 8, August 1996, Genetics Computer Group, 575 Science Drive, Madison, Wis., USA 53711) (Needleman, S. B. and Wunsch, C. D., (1970), Journal of Molecular Biology, 48, 443-453). Using GAP with the following settings for DNA sequence comparison: GAP creation penalty of 5.0 and GAP extension penalty of 0.3.

Nucleic acid molecules may be aligned to each other using the Pileup alignment software, available as part of the GCG program package, using, for instance, the default settings of gap creation penalty of 5 and gap width penalty of 0.3.

The nucleic acid molecules of the second aspect of the invention may also include variants capable of hybridising to the nucleic acid molecules of the invention, in particular the nucleic acid sequences defined in SEQ ID NOs:2, 4, 6, 8, 9 or 10 (and preferably SEQ ID NO.2) under conditions of low stringency, more preferably, medium stringency and still more preferably, high stringency and which encode a protein of the first aspect of the invention. Low stringency hybridisation conditions may correspond to hybridisation performed at 50° C. in 2×SSC.

Suitable experimental conditions for determining whether a given nucleic acid molecule hybridises to a specified nucleic acid may involve presoaking of a filter containing a relevant sample of the nucleic acid to be examined in 5×SSC for 10 min, and prehybridisation of the filter in a solution of 5×SSC, 5×Denhardt's solution, 0.5% SDS and 100 μg/ml of denatured sonicated salmon sperm DNA, followed by hybridisation in the same solution containing a concentration of 10 ng/ml of a 32P-dCTP-labeled probe for 12 hours at approximately 45° C., in accordance with the hybridisation methods as described in Sambrook et al. (1989; Molecular Cloning, A Laboratory Manual, 2nd edition, Cold Spring Harbour, New York).

The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridisation may be detected by exposure of the filter to an x-ray film.

Further, there are numerous conditions and factors, well known to those skilled in the art, which may be employed to alter the stringency of hybridisation. For instance, the length and nature (DNA, RNA, base composition) of the nucleic acid to be hybridised to a specified nucleic acid; concentration of salts and other components, such as the presence or absence of formamide, dextran sulfate, polyethylene glycol etc; and altering the temperature of the hybridisation and/or washing steps.

Further, it is also possible to theoretically predict whether or not two given nucleic acid sequences will hybridise under certain specified conditions. Accordingly, as an alternative to the empirical method described above, the determination as to whether a variant nucleic acid sequence will hybridise to, for example, the nucleic acid of SEQ ID NO:2, 4, 6, 8, 9 or 10, can be based on a theoretical calculation of the Tm (melting temperature) at which two heterologous nucleic acid sequences with known sequences will hybridise under specified conditions, such as salt concentration and temperature.

In determining the melting temperature for heterologous nucleic acid sequences (Tₘ₍ₕₑₜₑᵣₒ₎) it is necessary first to determine the melting temperature (Tₘ₍ₕₒₘₒ₎) for homologous nucleic acid sequence. The melting temperature (Tₘ₍ₕₒₘₒ₎) between two fully complementary nucleic acid strands (homoduplex formation) may be determined in accordance with the following formula, as outlined in Current Protocols in Molecular Biology, John Wiley and Sons, 1995, as:

Tₘ₍ₕₒₘₒ₎=81.5° C.+16.6(log M)+0.41(% GC)−0.61 (% form)−500/L

M=denotes the molarity of monovalent cations,

% GC=% guanine (G) and cytosine (C) of total number of bases in the sequence,

% form=% formamide in the hybridisation buffer, and

L=the length of the nucleic acid sequence.

Tₘ determined by the above formula is the Tₘ of a homoduplex formation (Tₘ₍ₕₒₘₒ₎) between two fully complementary nucleic acid sequences. In order to adapt the Tₘ value to that of two heterologous nucleic acid sequences, it is assumed that a 1% difference in nucleotide sequence between two heterologous sequences equals a 1° C. decrease in Tₘ. Therefore, the Tₘ₍ₕₑₜₑᵣₒ₎ for the heteroduplex formation is obtained through subtracting the homology % difference between the analogous sequence in question and the nucleotide probe described above from the Tₘ₍ₕₒₘₒ₎.

The polypeptides, nucleic acid molecules and antibodies of the present invention are “purified”. The term purified as used herein means altered “by the hand of man” from its natural state; i.e., if it occurs in nature, it has been changed or removed from its natural host and associated impurities reduced or eliminated. In one embodiment the object species is the predominant species present (i.e., on a molar basis it is more abundant than any other individual species in the composition). A substantially purified fraction includes a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

A third aspect of the invention provides a vector, such as an expression vector, that contains a nucleic acid molecule of the second aspect of the invention. The vectors of the present invention may comprise a transcription promoter, and a transcription terminator, wherein the promoter is operably linked with the nucleic acid molecule, and wherein the nucleic acid molecule is operably linked with the transcription terminator.

The vectors of the present invention may comprise further genes such as marker genes which allow for the selection of said vector in a suitable host cell and under suitable conditions.

The present invention further includes recombinant host cells comprising these vectors and expression vectors. Hence, a fourth aspect of the invention provides a host cell transformed with a vector of the third aspect of the invention. Illustrative host cells include bacterial, yeast, fungal, insect, avian, mammalian, and plant cells. Particularly preferred are cells such as E. coli which will express ohanin in a similar form as native ohanin (ohanin does not contain any post-translational modifications such as glycosylation or disulfide bridges).

A fifth aspect of the invention provides a method of producing a protein according to the first aspect of the invention, the method comprising culturing a host cell according to the fourth aspect of the invention under conditions suitable for the expression of the protein of the first aspect of the invention.

A sixth aspect of the invention provides a method of producing a protein according to the first aspect of the invention the method comprising the chemical synthesis of the protein by, for example, solid-phase peptide synthesis or combinatorial chemistry. Such techniques are well known in the art and will be readily able to be carried out by the skilled person.

The methods of the fifth and sixth aspect of the invention may further comprise the act of purifying the protein. Such methods are well known in the art and can be readily performed by the skilled person.

A seventh aspect of the invention provides a method of making an antibody which is capable of binding to a protein of the first aspect of the invention.

An eighth aspect of the invention provides an antibody which is capable of binding to a protein of the first aspect of the invention.

The antibodies of the invention may be polyclonal or monoclonal antibody preparations, monospecific antisera, human antibodies, or may be hybrid or chimeric antibodies, such as humanized antibodies, altered antibodies (Fab′)₂ fragments, F(ab) fragments, Fv fragments, single-domain antibodies, dimeric or trimeric antibody fragments or constructs, minibodies, or functional fragments thereof which bind to the antigen in question.

Antibodies may be produced using techniques well known to those of skill in the art and disclosed in, for example, U.S. Pat. Nos. 4,011,308; 4,722,890; 4,016,043; 3,876,504; 3,770,380; and 4,372,745. See also Antibodies—A Laboratory Manual, Harlow and Lane, eds., Cold Spring Harbor Laboratory, N.Y. (1988). For example, polyclonal antibodies are generated by immunizing a suitable animal, such as a mouse, rat, rabbit, sheep, or goat, with an antigen of interest. In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler & Milstein (1975) Nature 256:495-497, or a modification thereof. Typically, a mouse or rat is immunized as described above. Rabbits may also be used. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens).

The selected monoclonal antibody-secreting hybridomas are then cultured either in vitro (e.g., in tissue culture bottles or hollow fiber reactors), or in vivo (e.g., as ascites in mice).

Humanized and chimeric antibodies are also useful in the invention. Hybrid (chimeric) antibody molecules are generally discussed in Winter et al. (1991) Nature 349: 293-299 and U.S. Pat. No. 4,816,567. Humanized antibody molecules are generally discussed in Riechmann et al. (1988) Nature 332:323-327; Verhoeyan et al. (1988) Science 239:1534-1536; and U.K. Patent Publication No. GB 2,276,169, published 21 Sep. 1994).

An antibody is said to be capable of binding” a molecule if it is capable of specifically reacting with the molecule to thereby bind the molecule to the antibody.

Preferably, the antibody or fragment thereof has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹ or 10⁹ M⁻¹. The binding affinity of an antibody can be readily determined by one of ordinary skill in the art, for example, by Scatchard analysis (Scatchard, Ann. NY Acad. Sci. 51:660 (1949)).

A ninth aspect of the invention provides a method of producing a molecule, for example an antivenom against a protein according to the first aspect of the invention wherein the method comprises immunizing an animal with a protein according to the first aspect of the invention and harvesting antibodies from the animal for use as an antivenom.

Traditional methods of producing the treatment is to immunize a mammal such as a horse, goat or sheep against the venom. To reduce their toxicity, the venoms may be modified by treatment with formalin. To prolong their absorption, the modified venoms may be mixed with aluminum hydroxide gel. The antibodies thus produced are then isolated from the animal and used as an antidote in the patient, typically a human patient. More recently, non-mammals have employed using birds such as chickens. In this procedure, young chickens are immunized with small doses of the target-snake venom and as these animals grow older they develop antibodies which act as antidotes against the toxin. As the chickens become hens and start egg production, it has been found that the antivenom proteins are passed on, accumulating in the yolk. The eggs are then harvested for extraction of the proteins used to make the antidote.

The serum of the first animal (e.g. horse or chicken) is then administered to the afflicted animal (the “host”) to supply a source of specific and reactive antibody. The administered antibody functions to some extent as though it were endogenous antibody, binding the venom toxins and reducing their toxicity.

A tenth aspect of the invention provides an antivenom effective against a protein of the first aspect of the invention. The antivenom may be produced in accordance with the ninth aspect of the invention but the method of the eleventh aspect of the invention may also be used.

A further aspect of the invention contemplates the use of the proteins of the invention as a model for drug design and antivenoms. Accordingly, an eleventh aspect of the invention provides a method for identifying a modulator (e.g. an agonist or antagonist) compound of a polypeptide of the first aspect of the invention.

The polypeptides of the first aspect of the invention can be used to screen libraries of compounds in any of a variety of drug screening techniques. Such compounds may modulate (agonise or antagonise) the activity of a polypeptide of the first aspect of the invention.

In one embodiment, the method comprises contacting a test compound with a polypeptide of the first aspect of the invention and determining if the test compound binds to the polypeptide of the first or second aspect of the invention. The method may further comprise determining if the test compound enhances or decreases the activity of a polypeptide of the first or second aspect of the invention. Methods for determining if the test compound enhances or decreases the activity of a polypeptide of the first or second aspect of the invention will be known to persons skilled in the art and include, for example, docking experiments/software or X ray crystallography.

The polypeptide of the invention that is employed in the screening methods of the invention may be free in solution, affixed to a solid support, borne on a cell surface or located intracellularly.

Test compounds (i.e. potential modulators e.g. agonist or antagonist compounds) may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional minietics of the aforementioned.

Test compounds may be isolated from, for example, cells, cell-free preparations, chemical libraries or natural product mixtures. These modulators (e.g. agonists or antagonists) may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good modulators (e.g. antagonists or agonists) are molecules that bind to the polypeptide of the invention (in the case of antagonists without inducing the biological effects of the polypeptide upon binding to it).

Antagonists may alternatively function by virtue of competitive binding to a receptor for a polypeptide of the invention.

Agonists may alternatively function by binding to a receptor for a polypeptide of the invention and increasing the affinity of the binding between the receptor and the polypeptide of the invention.

Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the natural biological activity of the polypeptide is prevented.

It will be appreciated by those skilled in the art that the modulators and antagonists of the invention may find utility as an antivenom.

In certain of the embodiments described above, simple binding assays may be used, in which the adherence of a test compound to a surface bearing the polypeptide is detected by means of a label directly or indirectly associated with the test compound or in an assay involving competition with a labelled competitor.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

In silico methods may also be used to identify a modulator (e.g. an agonist or antagonist). The activity of the modulator (e.g. agonist and antagonist) moeities may then be confirmed, if desired, experimentally.

A twelfth aspect of the invention provides a pharmaceutical composition comprising a protein of the first aspect of the invention, a nucleic acid molecule of the second aspect of the invention, a vector of the third aspect of the invention, a host cell of the fourth aspect of the invention, an antibody of the eighth aspect of the invention, an antivenom of the tenth aspect of the invention, or a modulator (e.g. an agonist or antagonist) of the eleventh aspect of the invention.

The pharmaceutical compositions of the present invention may comprise a pharmaceutically acceptable carrier. The compositions may be administered alone or in combination with at least one other agent, such as a stabilizing compound, which may be administered in any sterile, biocompatible pharmaceutical carrier including, but not limited to, saline, buffered saline, dextrose, and water.

The pharmaceutical compositions utilized in this invention may be administered by any number of routes including, but not limited to, oral, intravenous, intramuscular, intra-arterial, intracerebroventricularly, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they can be placed in an appropriate container and labeled for treatment of an indicated condition. Such labeling may include the amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The dose ratio of toxic to therapeutic effects is the therapeutic index, and it can be expressed as the LD50/ED50 ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED50 with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art.

Where the polypeptides of the first aspect of the invention are used in pharmaceutical preparations/in medicine it may be desirable to use those polypeptides where the hyperalgesic function of the naturally occurring polypeptides has been reduced or abolished. Methods for achieving the same will be known to those skilled in the art and include, for example, site directed mutagenesis. To determine if a polypeptide exhibits reduced or absent hyperalgesic properties vis-à-vis ohanin (SEQ ID NO.1), this can, for example, be determined empirically by comparing the effects of the subject polypeptide and ohanin on mice in the hot plate assay as described in the Examples section below.

As discussed in more detail below, ohanin was found to exhibit significantly greater potency when administered via intracerebroventricular injection as opposed to intraperitoneally. Accordingly, in a one embodiment of the invention the moieties of the invention are administered directly to the nervous system e.g. via intracerebroventricular injection and may be formulated accordingly. In another embodiment of the invention the moieties of the invention may be administered intravenously.

A thirteenth aspect for a protein of the first aspect of the invention, a nucleic acid molecule of the second aspect of the invention, a vector of the third aspect of the invention, a host cell of the fourth aspect of the invention, an antibody of the eighth aspect of the invention, an antivenom of the tenth aspect of the invention, a modulator (e.g. an agonist or antagonist) of the eleventh aspect of the invention for use in medicine.

A fourteenth aspect of the invention provides for the use of a protein of the first aspect of the invention, a nucleic acid molecule of the second aspect of the invention, a vector of the third aspect of the invention, or a host cell of the fourth aspect of the invention of the twelfth aspect of the invention in the manufacture of a medicament for use as a sedative.

As mentioned above and described below in more detail, administration of ohanin to mice was found to render the mice sluggish and having reduced mobility. Accordingly, various moieties of the invention may find utility as a sedative.

Preferably, the medicament is for sedating warm-blooded animals such as pigs, cattle, humans and horses. Difficult problems arise because of the sensitivity of animals to stress situations. For example, dependent on such factors as breed, transporting conditions, weather and the like up to 5% pigs die during transport to slaughter houses because of their excitability. Losses can be even greater when the distances which animals need to be transported are great and which require several days or weeks, for example with transport of horses, cattle, sheep and pigs which are in these times transported over great distances by sea or air. Similar observations can also be made with chickens and also birds, for example exotic birds which are sometimes also transported over long distances to where they will be kept. States of excitement and aggressiveness associated therewith is probably also the reason for cannibalism in pigs which are kept in stalls. Larger animals, such as horses and cattle can cause significant problems because of their excitability not only when transported but also when being handled such as when being weighed.

For the above reasons, treatment of excitable animals, particularly horses, cattle and pigs, to calm states of excitement in stress situations, has been carried out with sedatives.

As mentioned above, where the moieties of the invention are employed for pharmaceutical purposes it will be generally desired to reduce or abolish the hyperalgesic effects of the ohanin protein.

When using the moieties of the invention as sedatives, the pharmaceutical compositions comprising the moieties may further comprise one or more additional sedatives.

A fifteenth aspect of the invention provides for the use of a protein of the first aspect of the invention, a nucleic acid molecules of the second aspect of the invention, a vector of the third aspect of the invention, a host cell of the fourth aspect of the invention, or a pharmaceutical composition of the twelfth aspect of the invention in the manufacture of a medicament for treating a neurological or muscular affliction.

As described below, ohanin may act directly on the central nervous system and induces hypolocomotion. These functions of ohanin may make it useful in the treatment of neurological or muscular afflications. Examples of such disorders which may usefully be treated in accordance with the present invention include Parkinsons disease, Huntingdon's Chorea, Epilepsy, bladder spasm, akathesia, Alzheimer's disease, amnesia, amyotrophic lateral sclerosis, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, paranoid psychoses, postherpetic neuralgia, schizophrenia, and Tourette's disorder. The present invention may also have applications in other fields where tremor or muscle spasm is present or is manifested—such as incontinence, asthma, brochial spasms, hic-coughs etc.

A sixteenth aspect of the invention provides a method of sedating an animal comprising administering a protein of the first aspect of the invention, a nucleic acid molecule of the second aspect of the invention, a vector of the third aspect of the invention, a host cell of the fourth aspect of the invention or a pharmaceutical composition of the twelfth aspect of the invention to the animal.

A seventeenth aspect of the invention provides a method of treating a patient with a neurological or muscular disease comprising administering to the patient a protein of the first aspect of the invention, a nucleic acid molecule of the second aspect of the invention, a vector of the third aspect of the invention, or a host cell of the fourth aspect of the invention.

An eighteenth aspect of the invention provides a defensive composition comprising a protein of the first aspect of the invention.

A defensive composition includes compositions which are used for personal defense purposes. The compositions of the eighteenth aspect of the invention may, for example, find utility for use against potential or actual personal attackers and made be used by members of the public or in law enforcement. In terms of the formulation of the defensive compositions of the eighteenth aspect of the invention guidance may be found above where the pharmaceutical compositions of the invention are discussed.

In one embodiment of the eighteenth aspect of the invention, the composition is provided in the form of a spray for ready administration to attackers etc.

Persons skilled in the art will be able to devise formulations which are readily absorbed and which, as such, would be suitable for use as a defensive composition where it is desired to quickly disable the attacker.

Both the ability of ohanin to induce pain and reduced mobility make it useful for defensive purposes. Moreover, as described below mice administered ohanin recovered with no obvious signs of paralysis or of hemorrhage or necrosis in the brains. Accordingly, the effects of ohanin appear to be reversible and as such make it particularly suitable in formulations for personal safety and law enforcement.

Whilst the invention has in certain places been described in relation to particular aspects of the invention the skilled reader will appreciate that the comments may apply equally to other aspects of the invention and the description should be construed accordingly.

The practice of the present invention will employ, unless otherwise indicated, conventional techniques of molecular biology, microbiology, and recombinant DNA technology which are within the skill of those working in the art. Such techniques are explained fully in the literature. Examples of texts for consultation include the following: Sambrook Molecular Cloning; A Laboratory Manual, Third Edition (2000) and subsequent editions.

### EXAMPLES

### Experimental Procedure

### Materials

Lyophilized king cobra crude venom was obtained from PT Venom Indo Persada (Jakarta, Indonesia). King cobra venom glands and liver were generously given by Dr Bryan G. Fry from Department of Biological Sciences, National University of Singapore, Singapore. The glands and liver were frozen immediately in liquid nitrogen and kept in −70° C. until used. All chemicals and reagents were purchased from Sigma (St. Louis, Mo., USA) with the exception of the following: Lys-C endopeptidase and trypsin were purchased from Wako Pure Chemicals (Osaka, Japan), reagents for Edman Degradation N-terminal sequencing (Applied Biosystem, Foster City, Calif., USA), acetonitrile (Merck KGaA, Darmstadt, Germany), Luria Bertani broth and agar were purchased from Q.BIOgene (Irvine, Calif., USA) and SDS-PAGE gel standards (Prestained broad range SDS-PAGE standards and Precision plus prestained dual-color standard) were purchased from Bio-Rad Laboratories (Hercules, Calif., USA). Superdex 30 Hiload (16/60) and μRPC C2/C18 (10μ 120 Å 2.1 mm×100 mm) columns were obtained from Amersham Pharmacia (Uppsala, Sweden). RP-Jupiter C18 (5μ 300 Å 1 mm×150 mm) and RP-Jupiter C18 (10μ 300 Å 10 mm×250 mm) columns were purchased from Phenomenex (Torrance, Calif., USA). Nickel-NTA agarose was purchased from Qiagen GmbH (Hilden, Germany). All the oligonucleotides were purchased from BioBasic (Shanghai, China) and 1^{st} Base Pte. Ltd. (Singapore). Platinum Taq polymerase, dNTP mix and ladders (50 bp, 100 bp and 1 Kb Plus) were purchased from GIBCO BRL® (Carlsbad, Calif., USA). All restriction endonucleases used were obtained from New England Biolabs® (Beverly, Mass., USA) and pGEMT-easy vector was obtained from Promega (Madison, Wis., USA). RNeasy® Mini kit, QIAGEN® OneStep RT-PCR kit, QIAprep® Miniprep kit, QIAEX II Gel Extraction kit and DNeasy® Tissue kit were purchased from Qiagen GmbH (Hilden, Germany). SMART™ RACE cDNA Amplification kit was purchased from Clontech Laboratories Inc. (Palo Alto, Calif., USA). ABI PRISM® BigDye™ Terminator Cycle Sequencing Ready Reaction Kit (ver 3.0) was purchased from PE Applied Biosystem (Foster City, Calif., USA). Water was purified with a MilliQ system (Millipore, Billerica, Mass., USA).

### Animals

Swiss albino male mice (20±2 g) were used for the animal experiments. In order to reduce the impact caused by environmental changes and handling during behavioral studies, mice were acclimatized to the Laboratory Animal Holding Center and laboratory surroundings for 3 days and at least 1 h prior to experiments, respectively. Animals were kept under standard conditions with food (low protein diet) and water available ad lib. The animals were housed 4 per cage in a light-controlled room (12 h light/dark cycle, light on 07:00 h) at 23° C. and 60% relative humidity. All behavioral experiments were performed between 08:30 h to 13:00 h. Each test group consisted of at least 7 mice and each mouse was used only once. All the animal experiments were conducted according to guidelines set by the Laboratory Animal Center of the National University of Singapore (adapted from Howard-Jones (10)).

### Liquid Chromatography-Mass Spectrometry (LC/MS) of King Cobra Venom

Lyophilized crude venom (60 μg) was dissolved in 20 μl of MilliQ water before loading via direct injection onto an RP-Jupiter C18 analytical column equilibrated with 0.1% (v/v) TFA (trifluoroacetic acid) attached to a Perkin-Elmer Sciex API300 LC/MS/MS system mass spectrometer (Thornton, Canada). The crude mixture was eluted using a linear gradient of 80% (v/v) ACN (acetonitrile) in 0.1% TFA at a flow rate of 50 μl/min. ESI/MS (Electrospray mass spectrum) data was acquired in positive ion mode with an orifice potential of 80 V. Nitrogen was used as curtain gas with a flow rate of 0.6/min and as nebulizer gas with a pressure setting of 100 psi. Full scan data was acquired over the ion range from 500 to 3000 m/z with step size of 0.1 Da. Data processing was performed with the aid of BioMultiview software (Perkin Elmer Sciex, Thornton, Canada).

### Isolation and Purification

Lyophilized crude venom (several batches of 200 mg each) was dissolved in 2 ml of MilliQ water and loaded onto a Superdex 30 column pre-equilibrated with 50 mM of Tris-HCl (pH 7.4). The proteins were eluted with 50 mM of Tris-HCl (pH 7.4) at a flow rate of 1 ml/min on a FPLC (Fast Protein Liquid Chromatography system, Amersham Pharmacia, Uppsala, Sweden). Protein elution was monitored at 280 nm. The fraction of interest was then loaded onto an RP-Jupiter C18 semi-preparative column equilibrated with 0.1% TFA (v/v) on Vision Workstation (PE Applied Biosystem, Foster City, Calif., USA). The bound proteins were eluted using a linear gradient of 80% ACN in 0.1% TFA (v/v) at a flow rate of 2 ml/min over an hour. Protein elution was monitored at 280 nm and 215 nm. The protein of interest was identified by mass determination (described below).

### Reduction and Pyridylethylation

Lyophilized and purified protein of interest was reduced and pyridylethylated using the procedure described earlier (11). Protein (500 μg) was dissolved in 500 μl of denaturant buffer (6 M GdnCl (Guanidine hydrochloride), 50 mM Tris-HCl, 1 mM EDTA pH 8.0). After the addition of 10 μl of β-ME (β-mercaptoethanol), the mixture was incubated at 37° C. for 2 h under vacuum. Subsequently, 20 μl of 4-vinyl pyridine was added to the mixture and kept at room temperature (˜25° C.) for another 2 h under vacuum. The reduced and pyridylethylated protein was loaded onto an RP-μRPC C2/C18 analytical column equilibrated with 0.1% TFA (v/v) on SMART Workstation (Amersham Pharmacia, Uppsala, Sweden). The bound proteins were eluted using a linear gradient of 80% ACN in 0.1% TFA (v/v) at a flow rate of 200 μl/min over an hour. Protein elution was monitored at 280 nm and 215 nm.

### Enzymatic Cleavage

Digestion of pyridylethylated protein with Lys-C endopeptidase and trypsin were performed at 37° C. for 20 h. Protein (150 μg) was dissolved in 150 μl of enzymatic digestion buffer (50 mM Tris-HCl, 4 M Urea, 5 mM EDTA pH 7.5) and proteases were added at a ratio of 1:50 (w/w).

### Chemical Cleavage

Digestion of reduced and pyridylethylated protein with formic acid (Asp-specific) was performed as described by Inglis (12). Briefly, 150 μg of pyridylethylated protein was dissolved in 2% of formic acid in a glass vial and then frozen at −30° C. Subsequently, under vacuum, the vial was thawed at room temperature and then sealed off. The vial was then heated at 108° C. for 2 h and allowed to cool to room temperature.

### Separation of Digested Peptides

The peptides generated by both the enzymatic and chemical digestions were fractionated using RP-μPC C2/C18 analytical column on SMART Workstation (Amersham Pharmacia, Uppsala, Sweden) using a linear gradient of 80% ACN in 0.1% of TFA (v/v) over an hour. The elution of peptides was monitored at 215 nm and 280 nm.

### Electrospray Ionization-Mass Spectrometry (ESI/MS)

ESI/MS was used to determine the precise masses and purity (±0.01%) of both the native protein and peptides. The RP-HPLC fractions were directly injected into the Perkin-Elmer Sciex API300 LC/MS/MS system mass spectrometer (Thornton, Canada). Ionspray, orifice and ring voltages were set at 4600 V, 50 V and 350 V, respectively. Nitrogen was used as curtain gas with a flow rate of 0.6 l/min and as nebulizer gas with a pressure setting of 100 psi. The mass was determined by direct injection at a flow rate of 50 μl/min using the LC-10AD Shimadzu Liquid Chromatography pump as solvent delivery system (40% ACN in 0.1% TFA). BioMultiview software was used to analyze and deconvolute the raw mass spectrum.

### Amino Terminal Sequencing

N-terminal sequencing of the native and digested peptides were performed by automated Edman degradation using a Perkin-Elmer Applied Biosystem 494 pulsed-liquid phase protein sequencer (Procise) with an on-line 785A PTH-amino acid analyzer. The derivatized PTH-amino acids were then sequentially identified by mapping the respective separation profiles with the standard chromatogram.

### Methods for Protein Administration

The volume injected via i.p. (intraperitoneal) route was 200 μl and the protein was dissolved in water. The i.c.v. (intracerebroventricular) injection was made in a volume of 2 μl through a puncture point at 1.5 mm lateral and 1.0 mm posterior to bregma using a 10 μl luer-tip Hamilton microsyringe with a modified needle so as to penetrate 2 mm from the top of the skull (13). The protein for i.c.v. injection was dissolved in ACSF (artificial cerebrospinal fluid). The needle was rotated on withdrawal. These two administration methods were used for the locomotor activity and hot plate experiments.

### In Vivo Toxicity Test

Native protein was injected i.p. into the mice at doses of 0.1 mg/kg, 1 mg/kg and 10 mg/kg (n=2). After injection, behavioral observations on the mice were recorded every 15 min for up to 6 h. Animals were sacrificed after 24 h and post-mortem examination was performed.

### Locomotor Activity

Locomotor activity of the mice was measured by an NS-AS01 activity monitoring system (Neuroscience, Inc., Tokyo, Japan), which is composed of an infrared ray sensor, a signal amplification circuit and a control circuit. Movement of the mice was detected by the infrared ray sensor on the basis of released infrared rays associated with their temperature. Each mouse was removed from its home cage and housed individually in a cage (12 cm×12 cm×30 cm) with an 8-channel infrared ray sensors placed over the cages. The cage contained approximately 40 ml of sawdust on the floor. Motor activity of eight animals kept in separate cages was measured simultaneously. All movements of a distance of 4 cm or more were detected by the infrared ray sensors and each represented a measure of general mobility of the injected mice. The activeness of the animals was assessed by performing a pre-run experiment. Animals used for the subsequent experiment had a minimum of 450 counts and a maximum of 850 counts over the first 20 min of the pre-run experiment. Active mice were then administered with the protein and placed in the same motor activity monitoring system. Immediately after this, counts of locomotor activity were collected in 10 min intervals for 80 min with a computer-linked analyzing system (AB System-24A, Neuroscience, Inc., Tokyo, Japan).

### Hot Plate Assay

Each mouse was placed on a hot plate (55° C.) and confined using a transparent plastic ring (diameter 12 cm, height 13 cm). The hot plate apparatus was a sealed wooden box with smooth metal surface 15 cm×15 cm and was heated using a water bath (Model Y22 Grant, Cambridge, UK). The latency time was measured from the time the mouse was gently introduced onto the hot plate to the time when it first showed one of the following responses: jumping, licking or stamping of a limb, as described by Woolfe and MacDonald (14). The hot plate assay was carried out 15 min after drug administration by i.p. or i.c.v. routes.

### Analysis of Results and Statistics

Changes in locomotor activity were analyzed by two-way ANOVA with repeated measures. Hyperalgesic effect induced by ohanin was analyzed using one-way ANOVA. All ANOVAs were followed by post-hoc analysis with Bonferroni correction. Statistical significance was indicated when p<0.05.

### Design, Assembly and Cloning of the Synthetic Gene

The full-length synthetic gene comprising of 369 bp was assembled from two fragments and each fragment was constructed from two overlapping oligonucleotides, ranging from 96 bp to 117 bp, respectively with an overlapping region of 21 bp enriched with more than 50% GC content to promote specific annealing. Primer 1 (5′-GGAATTCGTCGACGGATCCAT GGCTAGCCCGCCGGGTAACTGGCAGAAAGCGGACGTCACCTTCGATAGCAACACCG CGTTCGAAAGCCTGGTGGTGAGCCCGGAC-3′) and primer 2 (5′-TCC CCCCGGGCTGCCTAGGACGCACGGGCTCGAGGAGAAGCGTTCCGGGCTATCCGGCA CACCTTTCGGCACACCAACGTTTTCCACGGTTTTTTTGTCCGGGCTCACCACCAGGCT-3′) were used to prepare the first fragment; primer 3 (5′-TCCCCCCGGGTTTCCGTTCCGGAAAACACTTCTTCGAGGTGAAATACGGTACCCAGC GTGAATGGGCGGTGGGGCTAGCGGGTAAAAGCGTGAAGCGTAAGGGTTAC-3′) and primer 4 (5′-GACTAGTAAGCTTGCGGCCGCCTACAGCCACCACAGACCTTTCTGCCA GATACGTTCTTCCGCACCAGCCTTAAGTAACCCTTACGCTTCACGCT-3′) were used to prepare the second fragment. Nucleotides underlined were the flanking sequences for XmaI restriction site. PCR mixture to generate both the fragments contained a final concentration of 0.3 U Platinum Taq polymerase, 0.2 mM dNTP mix and 0.2 μM primers in a total volume of 25 μl. The amplification condition was as follows: 1 cycle of 94° C./1 min; 20 cycles of 94° C./30 s, 55° C./30 s, 72° C./1 min; and a final extension of 72° C./5 min. The two fragments were digested with XmaI and ligated together to obtain the full-length synthetic gene. This ligation product was cloned into pGEMT-easy vector and sequenced.

### Expression of Recombinant Ohanin

The 369 bp synthetic gene fragment was double digested by restriction endonucleases BamHI and NotI for cloning into the expression vector. VectorM (a modified version of pET32A) was used to express the synthetic ohanin in E. coli BL21/DE3 strain. The sub-cloning resulted in an expression of fusion protein consisting of hexahistidine tag at the N-terminal. For expression, a single colony harboring vectorM/ohanin, was inoculated into LB medium containing 100 μg/ml of Amp (ampicillin) incubated at 37° C. and 200 rpm for 14 h. The overnight culture was inoculated into fresh LB medium containing 100 μg/ml of Amp at 1:50 dilution. Again, the bacterial culture was incubated at 37° C. and 200 rpm until the culture reached an A₆₀₀ of approximately 0.6. Isopropyl-β-D-thiogalactopyranoside (IPTG) was then added to a final concentration of 0.1 mM to induce the expression and was further incubated at 16° C. and 200 rpm for 16 h before the bacteria were harvested. Bacterial cells were stored at −80° C. until used. The expression of recombinant protein in E. coli was analyzed by SDS-PAGE (sodium dodecyl sulfate polyacrylamide gel electrophoresis) according to the method of Laemmli (15) using 15% acrylamide gel.

The cells expressing the His-tagged fusion protein were thawed for 15 min and lysed using a final concentration of 0.5 mg/ml lysozyme at 4° C. for 15 min followed by mild sonication (six 1 min bursts) with adequate cooling in lysis buffer (10 mM Tris-HCl, 5 mM β-ME pH 8.0). After centrifugation at 16,000 rpm, the pellet containing the inclusion bodies and cell debris was collected and resuspended in binding buffer under denaturing conditions (6 M GdnCl, 10 mM Tris-HCl, 5 mM β-ME pH 8.0).

### Purification, Refolding and Cleavage of the Fusion Protein

The process of dissolution of the pellet was allowed to continue for at least 4 h at 4° C. using binding buffer under denaturing conditions. Cell debris that could not be dissolved by binding buffer was removed by centrifugation. The supernatant was loaded into a charged Ni-NTA resin column pre-equilibrated with binding buffer. Affinity chromatography was carried out according to manufacturer's guidelines. After washing extensively using wash buffer (10 mM IMD (Imidazole), 6 M GdnCl, 10 mM Tris-HCl, 5 mM β-ME pH 8.0), the bound protein was eluted using minimum volume of elution buffer (250 mM IMD, 6 M GdnCl, 10 mM Tris-HCl, 5 mM β-ME pH 8.0). Elution and concentration of the fusion protein was monitored at 280 nm

All the following refolding steps were carried out at 4° C. First, the concentration of the denatured fusion protein was adjusted using the elution buffer to approximately 6 mg/ml monitored at 280 nm. Then, 15 mg of the denatured fusion protein (2.5 ml) was diluted slowly in MilliQ water with 5 mM β-ME. MilliQ water containing 5 mM β-ME was delivered using a peristaltic pump (Amersham Pharmacia, Uppsala, Sweden) at a flow rate of 50 μl/min into the beaker containing the denatured fusion protein with vigorous stirring until the concentration of GdnCl slowly decreased to 1 M. The diluted unfolded fusion protein was dialyzed for 36 h against 200-fold excess MilliQ water containing 5 mM β-ME which was changed every 12 h.

Lyophilized refolded fusion protein (1 mg/ml) was dissolved in 0.1 M HCl. The solution was flushed with N₂ for 3 min prior to the addition of 100:1 molar excess of CNBr with respect to the Met content. Solutions of CNBr (Cyanogen bromide) were prepared fresh prior to experiment by dissolving the appropriate amount of solid in 100% ACN to a final concentration of 100 mg/ml. The reaction mixture was incubated at room temperature under darkness for 24 h before subjecting it to RP-HLPC for purification.

### Measurement of Circular Dichroism (CD) Spectra

The secondary structures of native and recombinant ohanin were measured by recording far UV CD spectra on a Jasco J810 spectropolarimeter (Jasco Corporation, Tokyo, Japan) with a 2 mm pathlength cell over a wavelength range of 260 nm to 190 nm at 22° C. The cuvette chamber was continuously purged with nitrogen before and during the experiments. Measurements for both the native and recombinant protein were made in MilliQ water and average of three scans taken to obtain a good signal to noise ratio. The results were expressed as the mean residue ellipticity (θ) in deg. cm².dmol⁻¹. The α-helix, β-sheet and random coil contents were estimated using the method described at http://www.embl-heidelberg.de/˜andrade/k2d/.

### Total RNA Isolation

Total RNA was isolated from king cobra venom gland according to the RNeasy® Mini kit manufacturer's protocol. Briefly, venom gland tissue (30 mg) was pulverised in liquid nitrogen using a mortar and pestle pre-cooled at −80° C. and further homogenized with 600 μl Buffer RLT using a Heidolph DIAX600 homogeniser (Schwabach, Germany). Completely homogenous lysate was obtained after 20 to 30 s and the lysate was centrifuged for 3 min at maximum speed. Cleared lysate was transferred to a new 1.5 ml eppendorf tube. 70% ethanol (550 μl) was added to the lysate and was mixed well by pipetting. The mixture was then loaded successively to an RNeasy mini spin column sitting on a 2 ml collection tube for centrifugation for 15 s at 13,000 rpm. Buffer RW1 (700 μl) was pipetted onto the RNeasy column for washing purposes and the column was centrifuged for 15 s at 13,000 rpm. RNeasy column was transferred to a new collection tube. Buffer RPE (500 μl) was used to wash the RNeasy column for 15 s at 13,000 rpm. Another 500 μl Buffer RPE was pipetted onto the RNeasy column and centrifuged for 2 min at maximum speed to dry the RNeasy membrane. RNeasy column was transferred to a new 1.5 ml eppendorf tube. RNase-free water (40 μl) was added directly onto the RNeasy membrane. After incubation at room temperature for 1 min, RNA was eluted from the membrane by centrifugation for 1 min at 14,000 rpm. The integrity of the RNA was examined by denaturing agarose gel electrophoresis.

### Reverse Transcriptase-Polymerase Chain Reaction (RT-PCR)

To generate gene-specific sequence, RT-PCR was performed using the QIAGEN® OneStep RT-PCR kit. In brief, RT-PCR mixture contained 2 μl of QIAGEN OneStep RT-PCR Enzyme Mix and the final concentration of 250 ng total RNA, 0.4 mM dNTP mix and 0.6 μM degenerate primers, respectively, in a total volume of 50 μl. Degenerate primers used were: RT1 sense primer 5′-GGNAAYTGGCARAARGCNGAY-3′ and RT2 antisense primer 5′-CCACCANARNCCYTTYTGCCA-3′. The reverse-transcription and amplification condition were: reverse-transcription at 50° C./30 min; initial PCR activation step at 95° C./15 min; immediately followed by 30 cycles of 3-step thermal cycling of denaturation at 94° C./1 min, annealing at 50° C./1 min, extension at 72° C./2 min; and a final extension at 72° C./10 min. The PCR products were then separated on a 1.5% TAE agarose gel by electrophoresis. The most intense bands were excised and purified before ligated into the pGEMT-easy vector. Inserts in the pGEMT-easy vector were sequenced on both strands with T7 and SP6 primers using the dideoxy chain termination method on an automated ABI PRISM® 3100 Genetic Analyzer (Applied Biosystem, Foster City, Calif., USA). ABI PRISM® BigDye™ Terminator Cycle Sequencing Ready Reaction Kit (ver 3.0) was used to carry out the cycle sequencing reaction. Analysis of the sequencing data was carried out using the Sequencing Analysis 3.7 (Sample Manager) software (Applied Biosystem, Foster City, Calif., USA).

5′- and 3′-Rapid Amplification of cDNA Ends (RACE)

The 5′- and 3′-RACE-Ready cDNA libraries were constructed using SMART™ RACE kit according to the manufacturer's protocol. For cDNA amplification, PCR reaction mix was prepared. The 5′-RACE reaction mix was consisted of 2.5 μl 5′-RACE Ready cDNA, 5 μl Universal Primer Mix (UPM) and a final concentration of 1.5 U Platinum Taq polymerase, 1.5 mM MgCl₂, 0.2 mM dNTP mix and 0.2 μM antisense primer (GSP2) (5′-CTTCCCAGCTAACCCAACAGCCCATTCCC-3′) in a total volume of 25 μl. The 3-step thermal cycling profile was as follows: 1 cycle of hot start at 94° C./1 min; 30 cycles of denaturation at 94° C./30 s, annealing at 67° C./30 s, extension at 72° C./2 min and followed by a final extension of 72° C./10 min. The 3′-RACE reaction mix, which yielded the full-length cDNA sequence, was consisted of 2.5 μl 5′-RACE Ready cDNA, 5 μl Universal Primer Mix (UPM) and a final concentration of 1.5 U Platinum Taq polymerase, 1.5 mM MgCl₂, 0.2 mM dNTP mix and 0.2 μM sense primer (GSP1) (5′-GATCATTTGATCCAGAGAAGACACAGTCTC-3′) in a total volume of 25 μl. The 3-step thermal cycling profile was as follows: 1 cycle of hot start at 94° C./1 min; 30 cycles of denaturation at 94° C./30 s, annealing at 68° C./30 s, extension at 72° C./3 min and followed by a final extension of 72° C./10 min. The PCR products were separated by 1.5% TAE agarose gel electrophoresis. The most intense bands were excised and purified before ligated into the pGEMT-easy vector. All full-length RACE clones were sequenced and assembled using the contig joining method available from DNAsis For Windows (ver. 2.5).

### Expression of Recombinant Pro-Ohanin

The cDNA, span the entire open reading frame excluding the signal peptide region, was added restriction sites at both ends using PCR. The primers used for introducing and amplifying the cDNA fragment were: sense primer (19K1) 5′-GTCGACGGATCCATGTCA CCTCCTGGGAATTGGCAG-3′ and antisense primer (19K2) 5′-AAGCTTGCGGCCGCT TAAAGATTTGCGAGTGAAACACG-3′. The PCR reaction mix contained the final concentration of 1.5 U Platinum Taq polymerase, 1.5 mM MgCl₂ 0.2 mM dNTP mix and 0.2 μM primers in a total volume of 25 μl. The 3-step thermal cycling profile was as follows: 1 cycle of hot start at 94° C./1 min; 30 cycles of denaturation at 94° C./1 min, annealing at 70° C./30 s, extension at 72° C./1 min followed by a final extension of 72° C./5 min. The gel purified PCR product was digested with restriction endonucleases BamHI and NotI for cloning into the expression vector. Expression vector, vectorM, was used to express pro-ohanin in E. coli BL21/DE3 strain. The sub-cloning resulted in an expression of fusion protein consisting of hexahistidine tag at the N-terminal region.

For expression, a single colony, harboring vectorM/pro-ohanin, was inoculated into LB medium containing 100 μg/ml of Amp cultivated at 37° C. with shaking at 200 rpm for overnight. The seed culture was inoculated into fresh LB medium containing 100 μg/ml Amp according to 1:50 dilution. Again, the bacterial culture was cultivated at 37° C. with shaking at 200 rpm until the culture reached an A₆₀₀ of 0.6. IPTG was added to a final concentration of 0.1 mM to induce the expression. The culture was further cultivated at 16° C. with shaking at 200 rpm before the bacterial cells were harvested. Bacterial cells were spun down at 6,000 rpm for 30 min. The cells expressing the His-tagged were lysed using a final concentration of 0.5 mg/ml lysozyme at 4° C. for 15 min, followed by mild sonication with adequate cooling in lysis buffer (10 mM Tris-HCl, 5 mM β-ME pH 8.0). After centrifugation at 16,000 rpm, supernatant was collected.

### Purification and Cleavage of Fusion Protein

The supernatant was loaded into a charged Ni-NTA resin column. Affinity chromatography was carried out according to manufacturer's guidelines. After washing extensively using wash buffer (10 mM IMD, 10 mM Tris-HCl, 5 mM β-ME pH 8.0), the bound protein was eluted using minimum volume of elution buffer (250 mM IMD, 10 mM Tris-HCl, 5 mM β-ME pH 8.0). Elution and concentration of the fusion protein was monitored at 280 nm

Freeze-dried and pure fusion protein was dissolved in MilliQ water at a concentration of 0.5 mg/ml. Stock thrombin was prepared to the concentration of 1 U/μl using MilliQ water. Thrombin was then added at the ratio of 1 U protease to 200 μg of recombinant protein and the cleavage reaction was continued for 16 h at 22° C. with gentle shaking. The expression and cleavage of recombinant protein in E. coli was analyzed by SDS-PAGE according to the method of Laemmli (15) using 15% acrylamide gel. The cleaved recombinant protein was separated from its fusion peptide and thrombin by RP-HPLC using an Jupiter C18 semi-preparative column. Identity and the precise molecular mass (±0.01%) of the recombinant protein were determined by Edman degradation sequencing and ESI/MS.

### Genomic DNA Isolation

Genomic DNA was isolated from king cobra liver tissue according to the DNeasy® Tissue kit manufacturer's protocol. Briefly, liver tissue (25 mg) was pulverised in liquid nitrogen using a mortar and pestle pre-cooled at −80° C. Tissue powder was transferred to a new 1.5 ml eppendorf tube. Buffer ATL and proteinase K of 180 μl and 20 μl, respectively, were added to lyse the cells. The lysate was incubated at 55° C. in a shaking water bath. After 3 h, 400 μg RNase A was added, mixed gently and incubated for 2 min at 16° C. to prevent RNA contamination. Buffer AL (200 μl) was added and mixed gently before incubating for 10 min at 70° C. 100% ethanol (200 μl) was added to the lysate to precipitate the genomic DNA. The mixture of lysate and white precipitates were loaded onto an DNeasy mini spin column sitting on a 2 ml collection tube for centrifugation for 1 min at 8,000 rpm. DNeasy spin column was transferred to a new collection tube. Buffer AW1 (500 μl) was used to wash the genomic DNA in the DNeasy column and was centrifuged for 1 min at 8,000 rpm. Another 500 μl of Buffer AW2 was used to wash the genomic DNA, before subjecting the spin column to another round of centrifugation at 14,000 rpm for 3 min to ensure that no residual ethanol was carried over during the following elution. DNeasy column was transferred to a new 1.5 ml eppendorf tube. Buffer AE (200 μl) was loaded directly onto the DNeasy membrane. After 1 min incubation at room temperature, genomic DNA was eluted from the membrane by centrifugation for 1 min at 8,000 rpm. The integrity of the genomic DNA was examined by 0.8% agarose gel electrophoresis.

### Genomic DNA PCR

For gDNA amplification, PCR reaction mix was prepared. The PCR reaction mix contained 1 μl gDNA as template and a final concentration of 1.5 U Platinum Taq polymerase, 1.5 mM MgCl₂, 0.2 mM dNTP mix and 0.2 μM primers in a total volume of 25 μl. The primers used were: sense (gDNA1) 5′-TCACCTCCTGGGAATTGG-3′ and antisense (gDNA2) 5′-AAG ATT TGC GAG TGA AAC-3′ as shown in FIG. 15A. The 3-step thermal cycling involved a hot start at 94° C./1 min followed by 30 cycles of 94° C./1 min, 60° C./30 s, 72° C./3 min; and a final extension of 72° C./10 min. The PCR product was analyzed on a 1.5% agarose gel and the band of interest was excised and purified. Sixteen clones carrying the inserts were sequenced on the both strands using the T7 and SP6 primers. All 16 clones were sequenced using additional internal primers to complete the sequence and assembled using the contig joining method available from DNAsis For Windows (ver. 2.5).

### Studies on Mechanism

The genes encoding ohanin and pro-ohanin used in these experiments were cloned into vector M and transformed into Escherichia coli strain BL21(DE3) as previously described. The purification was carried out according to the methods by Pung et al. (34) with slight modification. Purified N-terminal oligohistidine-tagged ohanin and pro-ohanin (termed His-ohanin and His-pro-ohanin respectively) were used directly for immunofluorerescence detection.

### In Vivo Administration of Ohanin and Pro-Ohanin

Swiss albino mice, weighing from 20 to 25 g, were used. Both the intracerebroventricular and intraperitoneal injections were performed using the methods described by Pung et al. (34). The animals were subsequently exsanguinated for surgical brain removal.

### Cryotomy

Mouse brains were extracted using lobotomic surgical techniques, and placed at 4° C. for 12 h in the solution containing 30% sucrose, 50 mM Tris-acetate, 5 mM EDTA (pH 7.4) and supplemented with complete protease inhibitor cocktail tablets (Roche).

The brain in the solution was then prepared for cryotomy using Leica CM840 cryotome pre-cooled at −25° C. The cryochuck was placed in the chamber and sufficient Optimal Cutting Temperature media (OCT) was added unto the chuck top until the media was almost frozen. The brain was then positioned laterally on its side and more OCT was added until the whole brain was covered by OCT and left to freeze over for 20 min. The chuck was then dipped into liquid nitrogen up to the bottom of the chuck top until the temperature was equilibrated. The brain was then placed into the cryotome and 10 μm slices were cut and placed unto Superfrost Plus (Menzel-Gläser) slides. The slides were stored in −20° C. Prior to any assays, the slides were placed overnight in 0.01% BSA dissolved in phosphate-buffered saline (PBS) at 4° C. to prevent non-specific binding of proteins and antibody onto the slide surface.

### In Vitro Binding Assays

The proteins were dissolved in PBS at a concentration of 0.05 μM. Protein solutions (1 ml) were then placed unto each slide with the uninjected brain slices, and incubated overnight at 4° C. The slides were then rinsed by placing them in a rocking incubator with ice-cold PBS and subjected to rocking on a rocking incubator at RT for 30 min. For the competition assay, 1 ml of the second protein solution was added unto each slide and re-incubated overnight at 4° C. after the first rinse step, and the rinsing procedure was repeated.

Two additional washes were performed using ice-cold PBS after the rinsing step on a rocking incubator at RT with 15 min each wash.

### Immunofluorescence

Rabbit anti-His antibody (α-His, goat) from Anaspec Inc was used at the ratio of 1:1000 in PBS. The antibody solution (1 ml) was placed unto each prepared slide and incubated overnight at 4° C. The slides were then washed 3× by placing them in a rocking incubator in ice-cold PBS and further subjected to rocking on a rocking incubator at RT for 15 min. Anti-rabbit secondary antibody conjugated with Alexa-fluo488 from Molecular Probes was used at the ratio of 1:500. Secondary antibody solution was added unto the slides and incubated overnight in dark at 4° C. The slides were washed as with the primary antibody wash in the dark, and left to semi-dryness. Prolong Gold Antifade mounting media with DAPI (Molecular Probes) was then added unto the slides and a coverslip was placed over. The brain slices were viewed with the Zeiss Axiovert 200M microscope with a Axiocam HRc digital camera attachment. The subsequent pictures were taken with the Axiovision ver 4.3.0.101 at 405 and 480 nm wavelengths. They were edited and overlayed using Photoshop version 5.5.

### Results

Identification of Novel Protein from King Cobra Venom

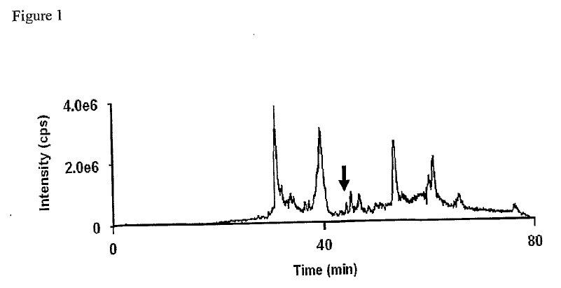

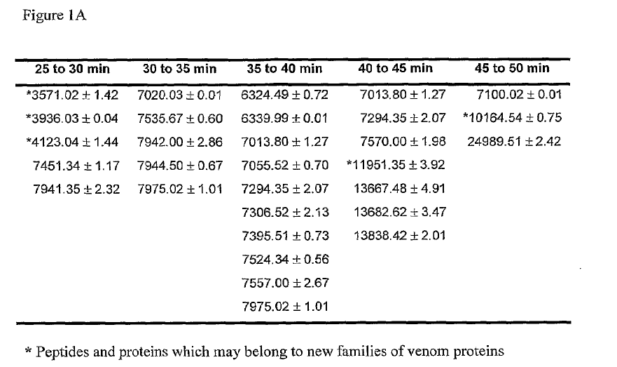

Crude venom from Ophiophagus hannah (King cobra) was profiled using LC/MS (FIG. 1) to identify new and interesting protein components in the venom. Peptides and proteins detected by LC/MS were organized by retention time (FIG. 1A). Proteins eluted after 50 min gave a relatively noisy m/z spectra and hence their molecular masses were not determined. This could be due to the large size of the proteins as well as the glycosylation and other post-translational modifications. Thus, mass profiling of king cobra venom using LC/MS demonstrates the ineffectiveness and limitation of this technique. With the LC/MS profile, we first searched for proteins with masses that are distinct from that of the well-established toxin families. We identified a protein with a molecular mass of 11951.35±3.92 Da which was different from any of the established families and hence we decided to carry out further studies on this novel protein.

### Isolation and Purification of the Novel Protein

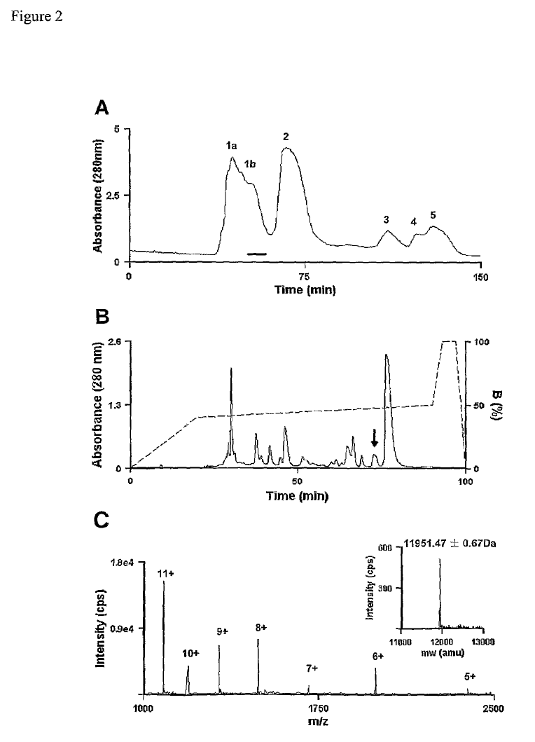

The novel protein was purified from king cobra venom via a two-step purification procedure. The first step involved the separation of the crude venom using gel filtration chromatography. Since the molecular weight of the novel protein was approximately 12 kDa, Superdex 30 (Hiload 16/60) column was selected for gel filtration chromatography. Gel filtration of crude venom yielded five major peaks (FIG. 2A). We subjected the first three peaks from gel filtration chromatography to RP-HPLC. Individual fractions from RP-HPLC were assessed using ESI/MS (data not shown). The protein fraction, which eluted at a gradient of 38 to 40% buffer B (80% ACN in 0.1% TFA) (FIG. 2B) from Peak 1b of gel filtration, was found to be homogenous with a molecular mass of 11951.47±0.67 Da (FIG. 2C). The overall yield of the novel protein was approximately 1 mg from 1 g of crude venom.

### Determination of the Amino Acid Sequence

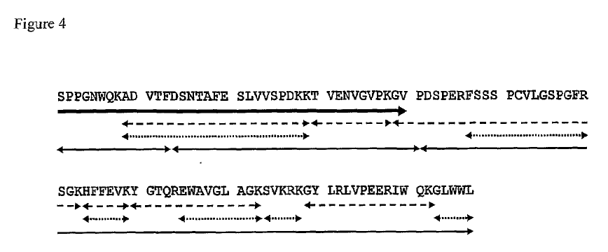

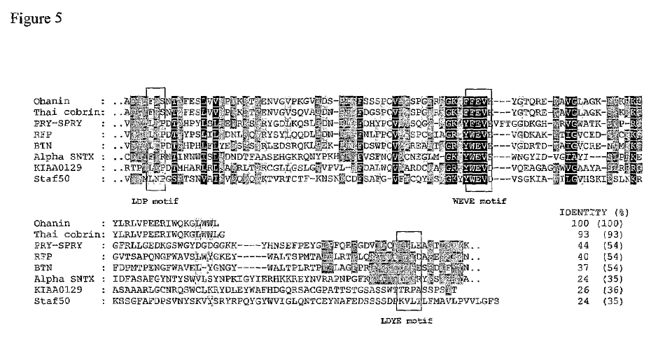

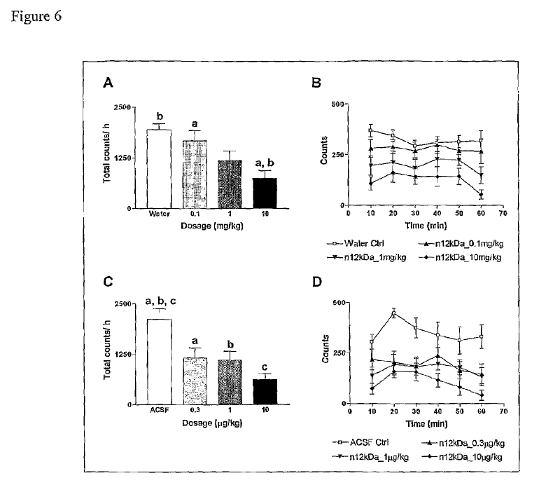

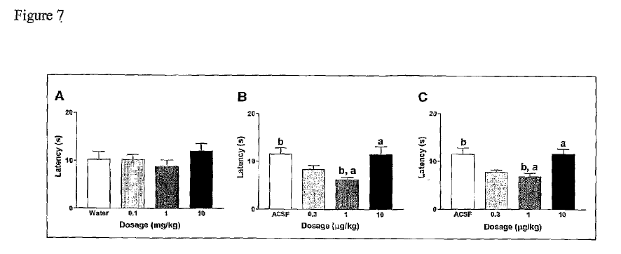

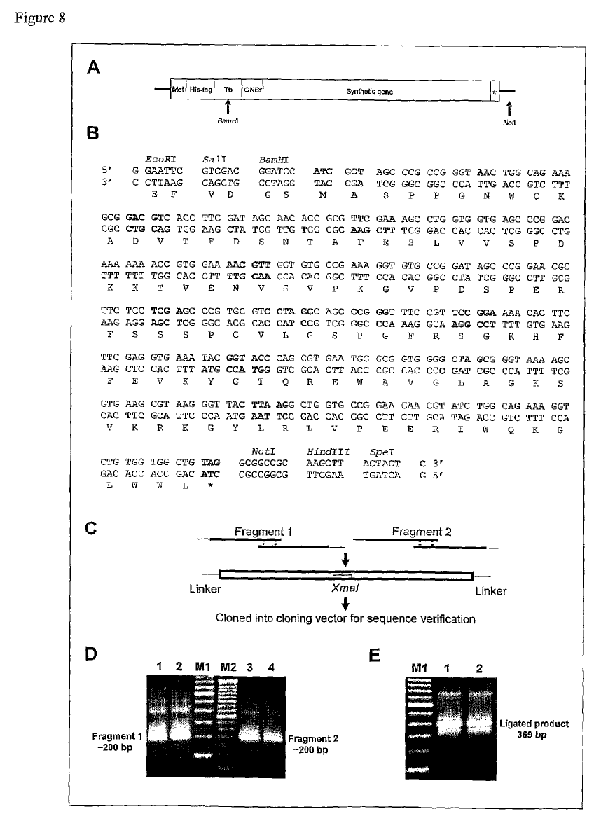

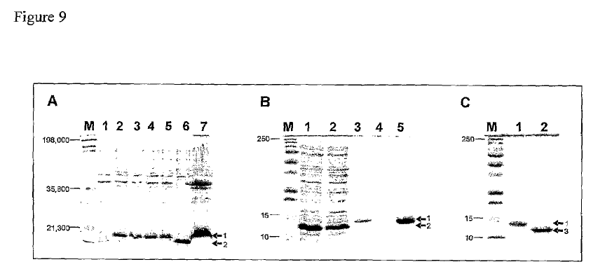

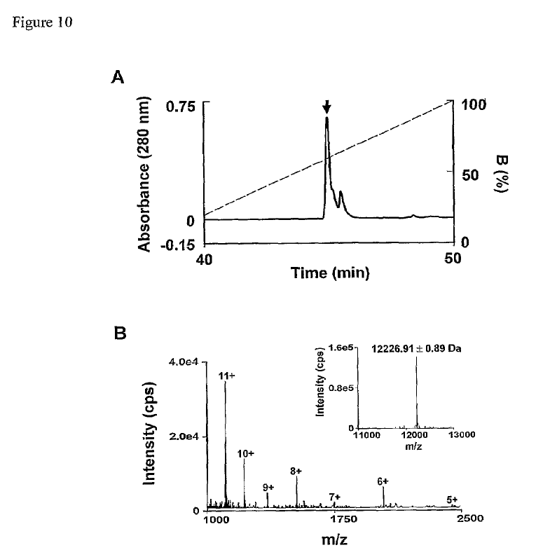

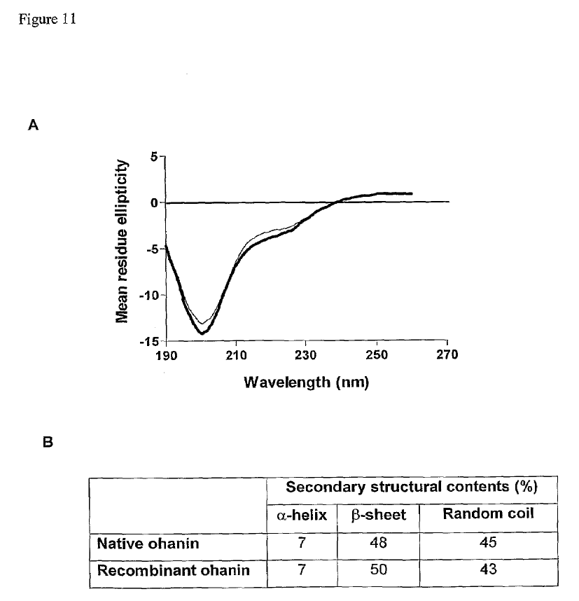

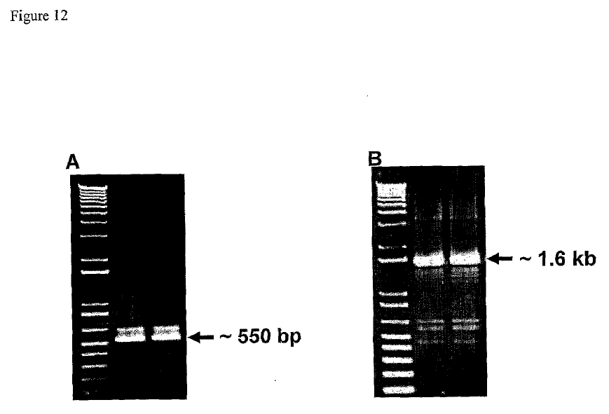

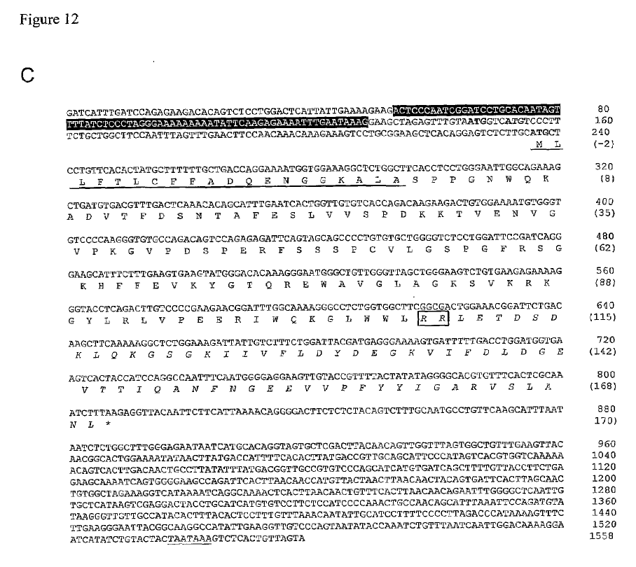

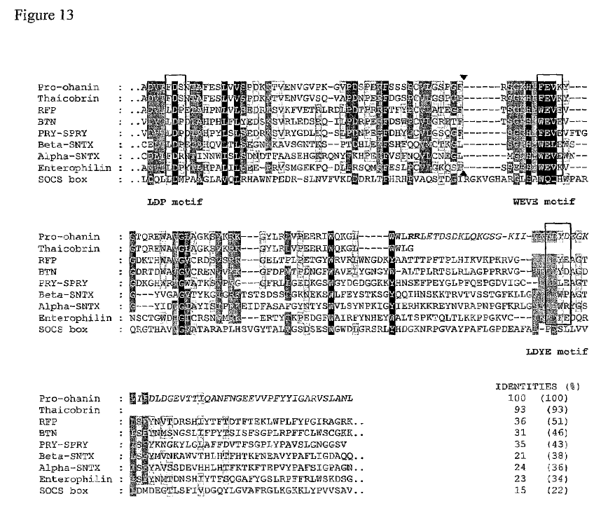

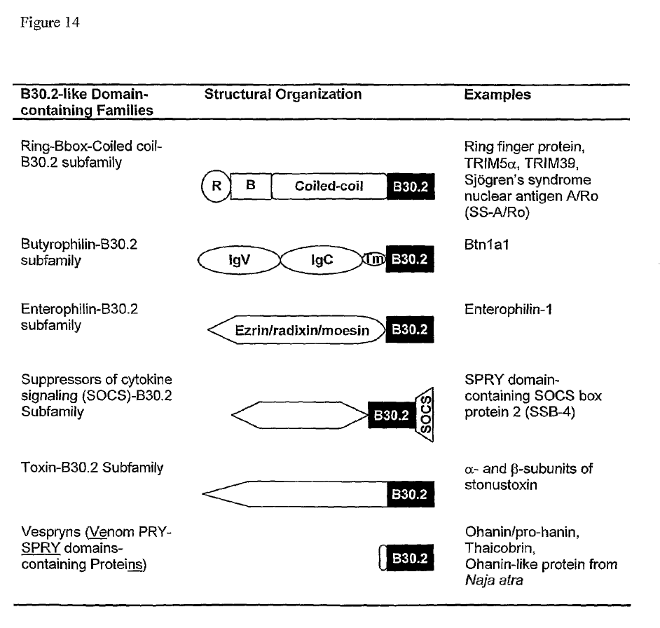

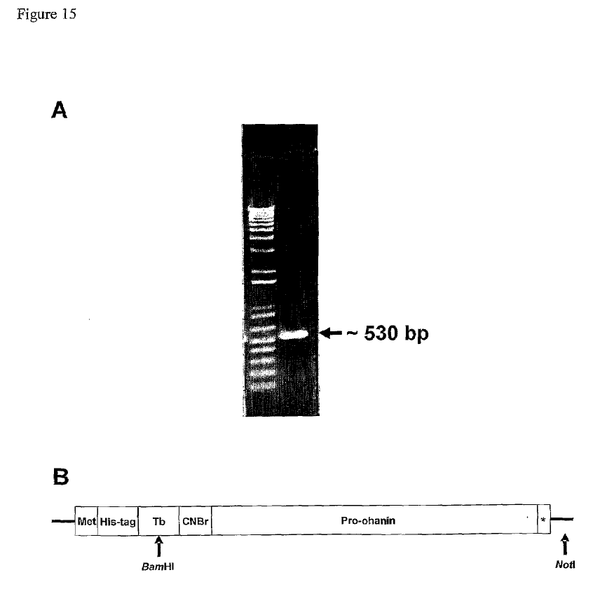

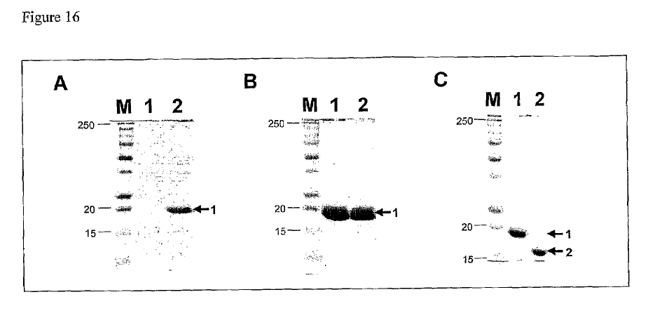

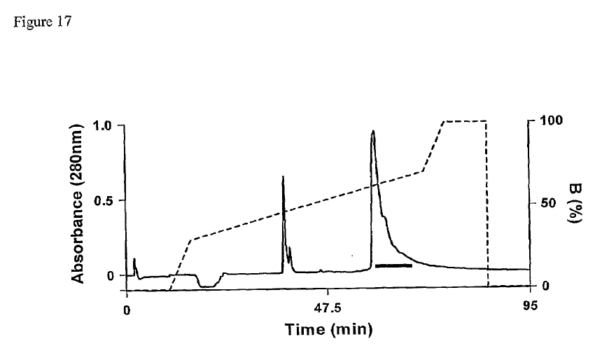

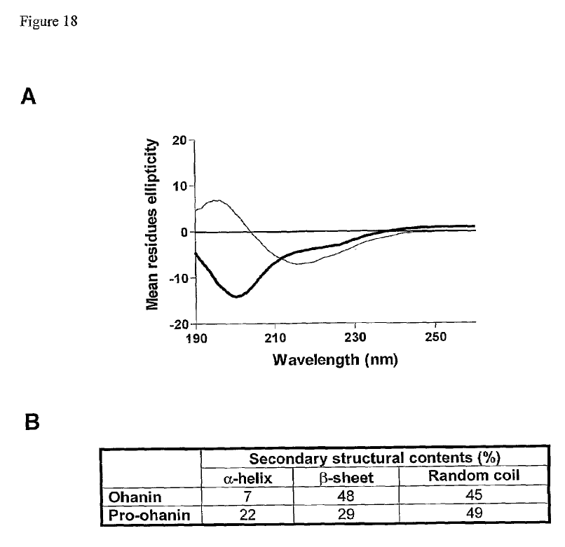

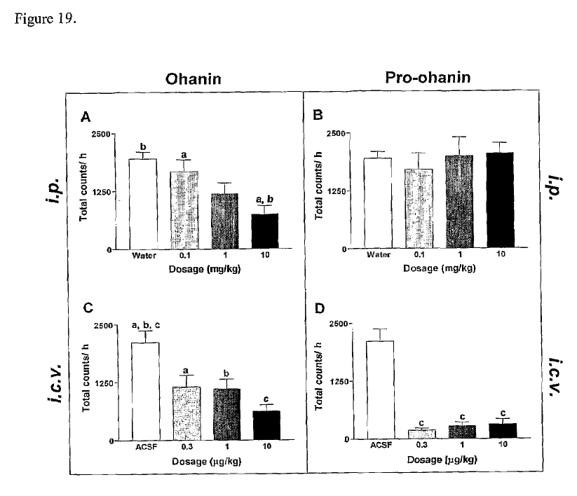

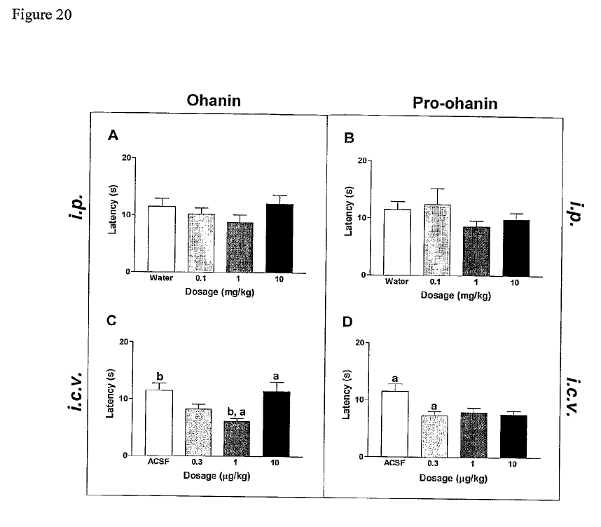

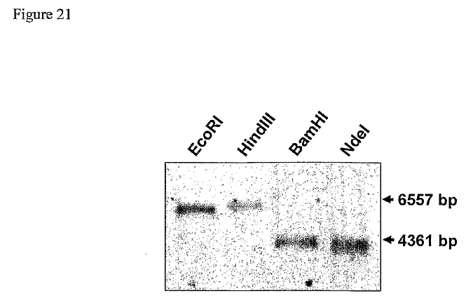

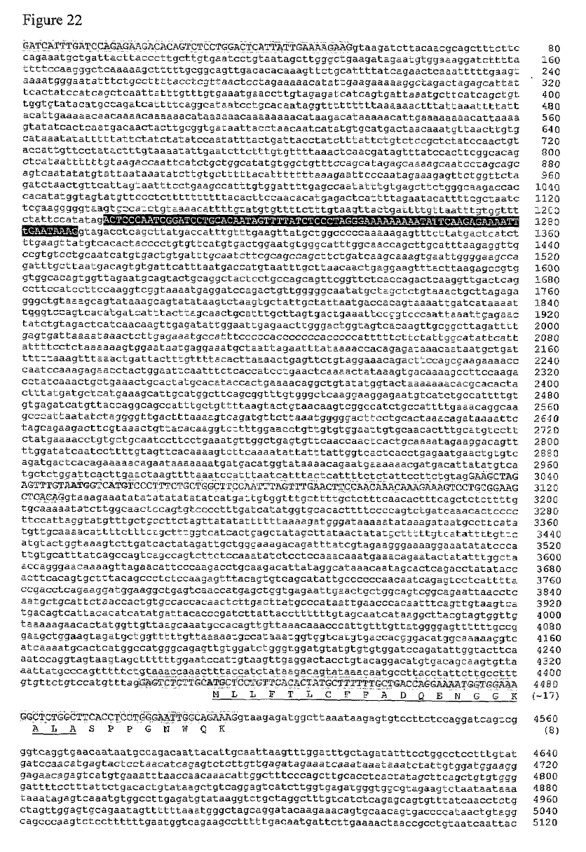

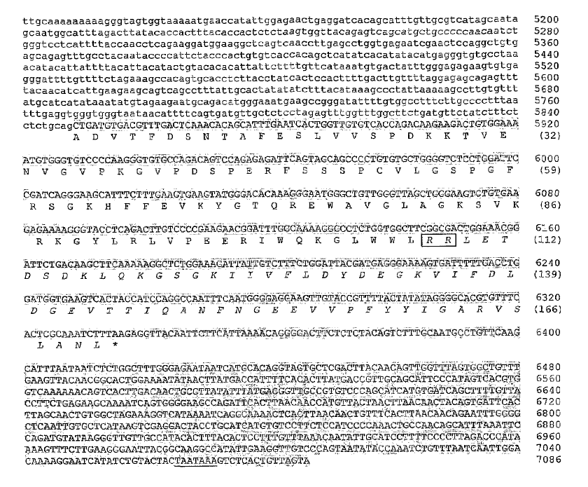

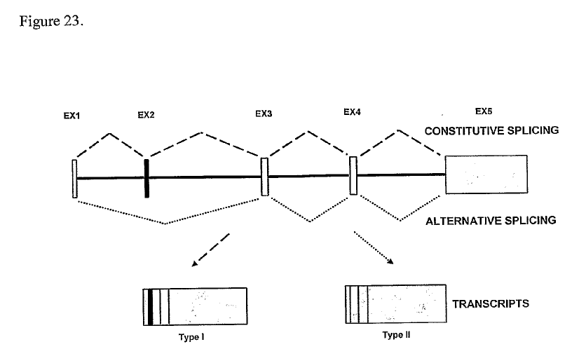

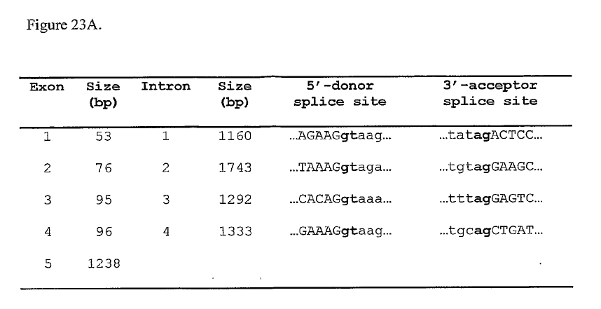

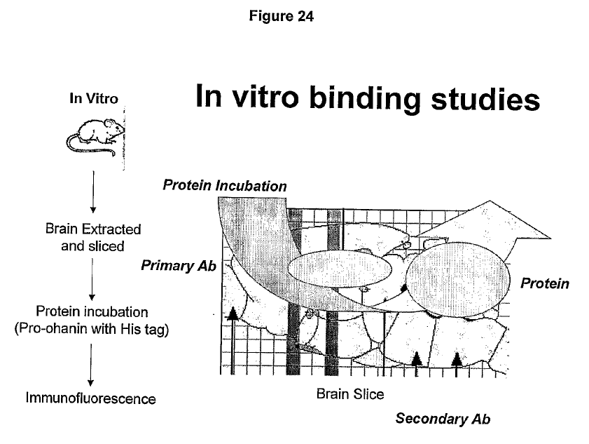

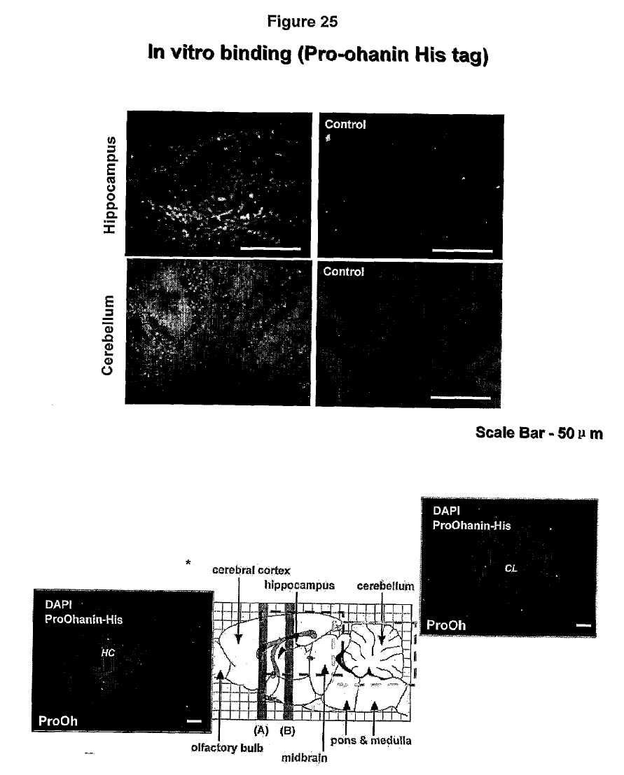

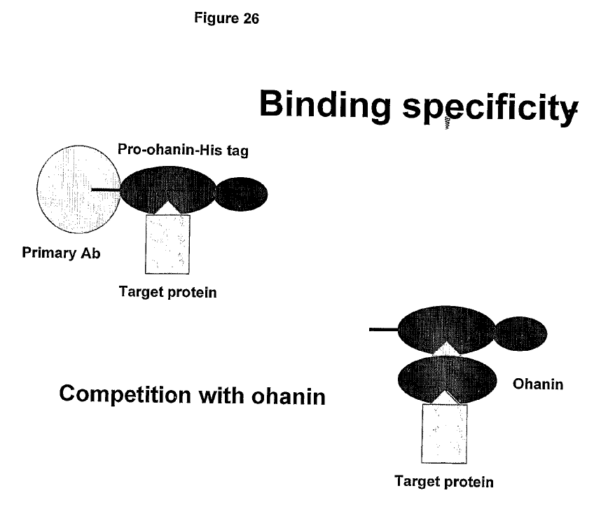

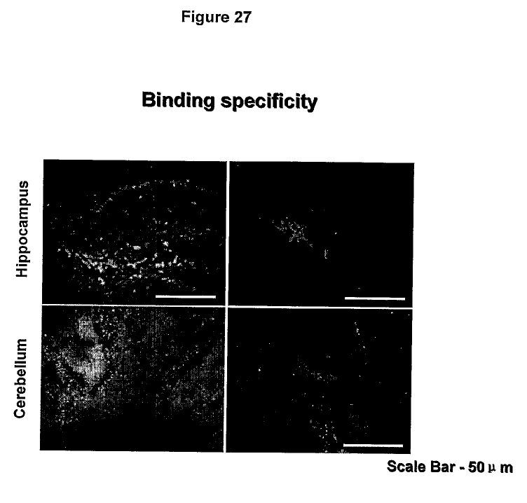

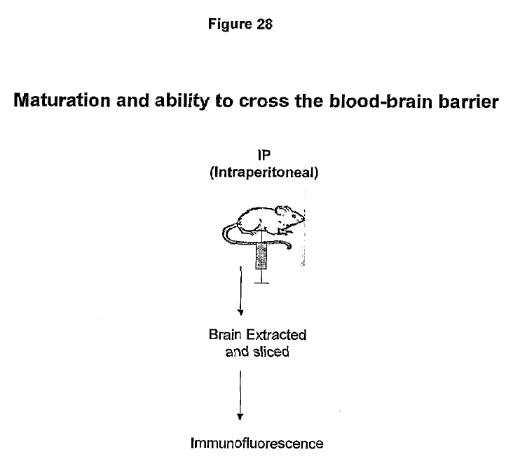

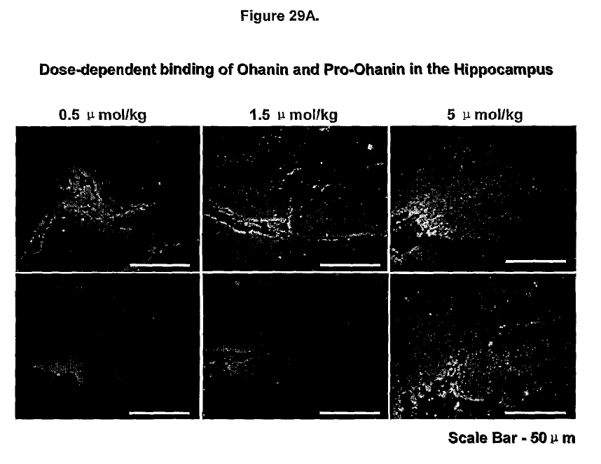

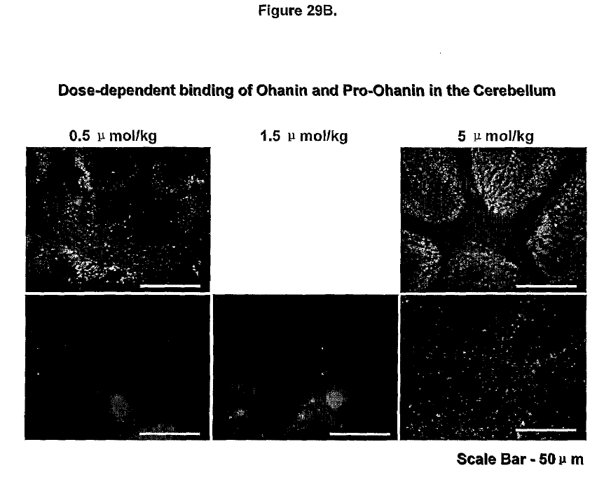

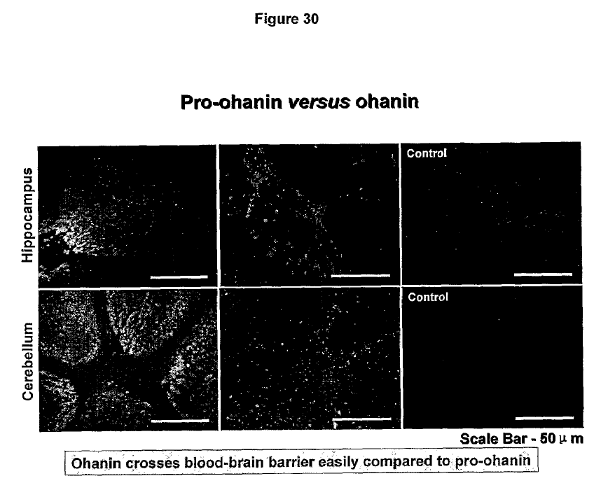

N-terminal sequencing of the native protein was determined by Edman degradation and it resulted in the identification of the first 40 residues. The N-terminal sequence showed no sequence homology to any of the proteins from known snake toxin families. To complete the sequence, pyridylethylated protein was digested with Lys-C endopeptidase, trypsin and formic acid. Peptides from the respective digests were separated by reverse phase HPLC (FIG. 3). Molecular mass and the amino terminal sequences of the purified peptides were obtained to complete the full-length amino acid sequence (FIG. 4). The sequences of peptides and the entire protein were verified by comparing the calculated and observed masses of the digested peptides (FIG. 3D). The observed molecular masses matched well with the calculated molecular masses. The novel protein contains 107 amino acid residues with one free cysteine and no post-translational modifications. We named this novel protein as ohanin because it was purified from the venom of king cobra Ophiophagus hannah.

### Sequence Analyses of Ohanin

Comparison of the full length amino acid sequence of ohanin with those of other proteins using the BLASTP algorithm (http://www.ncbi.nlm.nih.gov/BLAST/) (16) showed 93% sequence identity with That cobrin (SP: P82885) isolated from monocled cobra (Naja kaouthia). Although its sequence was deposited in the protein database, there is no published literature on Thai cobrin. Thus, ohanin and That cobrin form the first members of a new family of snake toxins. Other than with That cobrin, ohanin did not display significant sequence similarity (E-values>10⁻⁵) with other proteins in the GenBank (database.

As a second step, Conserved Protein Domain Database (CDD) (http://www.ncbi.nlm.nih.gov/Structurelcdd/wrpsb.cgi) (17) was used to search for conserved domains to predict the biological function of ohanin, based on the assumption that domains are the fundamental units of protein structure and function (18). Residues 9 to 107 of ohanin displayed an overall identity of 44% and similarity of 54% to the truncated PRY-SPRY domains (FIG. 5). PRY is a domain which is present in tandem with SPRY domain (For details, see discussion). The SPRY domain has been identified as a sub-domain within the B30.2-like domain (19). SPRY domains and B30.2-like domain are found in a variety of proteins (For details, see Henry et al. (20, 21)).

### In Vivo Toxicity Test

Ohanin was used for in vivo toxicity study in mice. All mice were observed to be active prior to the start of the experiment. Upon i.p. injection of the protein, it was observed that mice with doses of 1 mg/kg and 10 mg/kg became quiet and sluggish. However, they recovered 2 h after the injection with no obvious signs of paralysis of the limbs and respiratory system. None of the mice died even at doses of 10 mg/kg. Analysis of gross pathology 24 h after the injection showed no signs of hemorrhage or necrosis in the brain, heart, lungs, kidneys, spleen and liver as compared to those from the control animals (data not shown).

### Locomotor Activity

To quantitatively verify our observations from the in vivo toxicity test, effects of ohanin on the locomotor activity of the injected mice were examined. As shown in FIG. 6A, ohanin at doses of 0.1 mg/kg, 1 mg/kg and 10 mg/kg induced dose-dependent hypolocomotion after i.p. injection (F_{3,30}=5.787, p<0.01). The decrease in the locomotor activity was statistically significant between 10 mg/kg dose and the 0.1 mg/kg dose (p=0.030); between 10 mg/kg dose and the control (p=0.004) as shown in FIG. 6A. At 10 mg/kg dose the total movement counts decreased to 742±190 compared to the controls (1942±147). The dose-dependent inhibition was different even at 10 min after injection as shown in FIG. 6B. There was no statistically significant time effect within the same dose for the whole experimental duration of 1 h as indicated by two-way repeated measures ANOVA, suggesting that the inhibition effect was not yet recovered. The effect beyond the experimental duration (1 h) was not determined. Prolonged physical activity and lack of food might exhaust the mice. Hence, the resulting sluggishness beyond the experimental duration may not be representative of the protein's effects alone.

Intracerebroventricular injection was used to assess the direct effect of ohanin on the central nervous system. The dosages used for i.c.v. were approximately 1000-fold less than that given for i.p. of the high, intermediate and low doses. Ohanin showed statistically significant (F_{3,26}=9.112, p<0.001) and dose-dependent hypolocomotion as shown in FIG. 6C with p values of 0.027, 0.009 and 0.000 at doses of 0.3 μg/kg, 1 μg/kg, and 10 μg/kg, respectively, as compared to the control. Even at 0.3 μg/kg dose the total movement counts decreased to 1155±248 compared to that of the control mice (2109±264). In addition, the onset of response decreased in a dose-dependent manner immediately after injection and lasted for an hour (FIG. 6D). There was no significant time effect within the same dose for the whole experimental duration. This was similar to the results obtained from i.p. injection but at extremely low doses. The IC₅₀ (dose needed to reach ˜50% inhibition of the locomotion counts) values for i.p. and i.c.v. injections were 3.25 mg/kg and 0.5 μg/kg, respectively. Thus ohanin exhibits high potency in inducing hypolocomotion at ˜6,500-times lower doses when injected through i.c.v. route, suggesting a central nervous system pathway in the observed effect on locomotion.

### Hot Plate Assay

The nociception caused by thermal pain stimulus to the ohanin-administered mice was assessed using the hot plate assay. The dosages used in hot plate assay were the same as those used for the locomotor activity. Effect of ohanin on the pain stimulus was evaluated 15 min after i.p. and i.c.v. injections. As shown in FIGS. 7A and 7B, both the i.p. and i.c.v. injections induced a similar U-shaped dose-response curve. There were no significant effects at all the dosages used when ohanin was injected i.p. (F_{3,28}=0.867, p>0.05) (FIG. 7A). However, it showed a dose-dependent hyperalgesic effect when injected i.c.v. at doses of 0.3 μg/kg and 1 μg/kg (F_{3,50}=6.390, p<0.01). But at higher dose of 10 μg/kg, there was no significant hyperalgesic effect. The latency time was statistically significant between 1 μg/kg and the control (p=0.002); between 1 μg/kg and 10 μg/kg (p=0.015) as shown in FIG. 7B.

### Design, Assembly and Cloning of the Synthetic Gene

Since the natural abundance of ohanin is low in the crude venom, a synthetic gene that encodes for ohanin based on its protein sequence was constructed by recursive PCR method (22). The E. coli expression system was selected as ohanin does not contain any post-translational modifications such as glycosylation or disulfide bridges. Secondly, over-expression of ohanin in E. coli expression system has the advantage of providing adequate amount of recombinant protein to facilitate our future studies on its structure-function relationships.

The overall strategy for synthetic gene design and construction are shown in FIG. 8 (For details, see also Discussion). FIG. 5A shows the synthetic gene construct for the expression in vectorM. FIG. 8B shows the reverse-translated DNA sequence of the full-length synthetic gene. The strategy for generation of overlapping oligonucleotides in order to obtain the 369 bp synthetic gene is shown in FIG. 8C. Two pairs of oligonucleotides were used to assemble the two fragments (P1 and P2). These two fragments were then ligated via the XmaI site to generate the entire gene. PCR reaction for the extension of overlapping oligos to generate fragments 1 and 2 was performed using the two pairs of oligonucleotides (FIG. 8D). Ligation of the fragments via XmaI restriction site yielded the full-length synthetic gene of 369 bp (FIG. 8E). The synthetic gene was cloned into the pGEMT-easy vector and sequenced on both strands with T7 and SP6 primers before sub-cloning into the expression vector.

### Expression of Recombinant Ohanin

E. coli harboring vectorM/ohanin construct was used for the expression of recombinant ohanin. SDS-PAGE analysis of total protein prepared from bacterial culture after overnight induction using 0.1 mM IPTG at 16° C. demonstrated an abundant protein of apparent molecular mass of approximately 14 kDa. Comparison of total proteins extracted from uninduced and induced cultures together with fractionation of fusion protein into soluble and insoluble proteins are shown in FIG. 9A. An intense band of 14 kDa (indicated with arrow as 1) corresponding to fusion protein appeared in the insoluble fraction. No significant differences in expression of the recombinant protein were observed on changing various parameters, such as expression vectors, bacterial strains, cell density in the culture, incubation temperature, buffers and the amount of IPTG used (data not shown).

### Purification and Cleavage of Fusion Protein

The His-tag in the fusion protein allowed for rapid purification using a single affinity column under denatured condition. The purification steps are shown in FIG. 9B. Lane 3 showed one major species (˜14 kDa) and Lane 5 shows the fusion protein with an apparent molecular mass of 14 kDa after refolding. The additional 2 kDa of the recombinant protein as compared to the native one corresponds to the N-terminal His-tag, thrombin and CNBr cleavage sites (FIG. 8A). From 1 l of bacterial culture, 25 mg of His-tagged fusion protein was purified using Ni-NTA affinity chromatography.

CNBr was used to cleave the His-tag from the recombinant protein (FIG. 9C). After cleavage, the recombinant ohanin was purified using RP-HPLC (FIG. 10A), and molecular mass and homogeneity of the protein were determined by ESI-MS. The recombinant ohanin was homogenous, with a molecular mass of 12226.91±0.89 Da as assessed by ESI/MS (FIG. 10B). The identity of the recombinant protein was further confirmed using N-terminal sequencing by Edman degradation. The N-terminal sequence of the recombinant ohanin was ASPPG, which corresponds to the N-terminal of the native protein except for the alanine that was inserted to improve the efficiency of CNBr cleavage.

### Characterization of Recombinant Ohanin

The secondary structures of the native and recombinant protein were evaluated by CD spectroscopy analysis (FIG. 1A). The CD spectrum of the native protein showed negative ellipticity extrema near 200 nm and 215 nm, indicating a β-sheet and random coil structures with more of β-sheet conformation. The CD spectrum of the recombinant protein is similar to that of the native protein with negative ellipticity values at 200 nm and 215 nm. The constitutions of the secondary structures calculated from the CD spectra are shown in FIG. 11B.

To test whether recombinant ohanin has similar pharmacological actions as the native protein, we studied its hyperalgesic effect in i.c.v. administered mice. The recombinant ohanin exhibited the U-shaped dose-response curve similar to that of the native protein (F_{3,45}=5.783, p<0.01) (FIGS. 7B and 7C). Significant differences were found between 1 μg/kg dose and the control; and between 1 μg/kg and 10 μg/kg dose with p value of 0.007 and 0.015, respectively. These results indicate that the recombinant ohanin is structurally and functionally similar to the native protein isolated from the venom.

### Cloning and Sequencing of Ohanin

The total RNA extracted from the king cobra venom gland was low (˜4 μg each extraction), but the quality of RNA was relatively good. We used a combination of RT-PCR and RACE techniques to obtain the full-length cDNA of ohanin. To isolate gene specific sequences, RT-PCR was first performed using the total RNA as template. Degenerate primers, RT1 and RT2, were designed based on its known amino acid sequence. The amplified fragments, ˜200 bp in size, were gel purified, ligated into the pGEMT-easy vector and sequenced. Sequencing analysis revealed that all eight clones encoded for the amino acid sequence with complete homology to partial ohanin sequence.

Next, the 5′-coding region together with its 5′-UTR (untranslated region) were amplified using UPM (Universal Primer Mix) and an antisense primer, GSP2. Two bands, 550 and 600 bp respectively, were obtained from the 5′-RACE amplification (FIG. 12A). However, only 550 bp band gave the expected coding sequence of ohanin. We further designed a sense primer, GSP1, from the beginning of the 5′-UTR sequence. The 3′-RACE amplification was performed using GSP1 and UPM, which in turns yielded the full-length cDNA sequence of 1558 bp (FIG. 12B). The full-length cDNA sequence of ohanin and its deduced amino acids sequence are shown in FIG. 12C.

The cDNA encodes for a putative open reading frame of 190 amino acids. It was flanked by 234 bp of 5′-UTR and 783 bp of 3′-UTR including the poly-A tail. Interestingly, the putative open reading frame encodes for an extra of 63 amino acids to the C-terminal of the mature ohanin. This is the first cDNA sequence reported so far from snake origins that carried a pro-peptide segment at the C-terminal of the mature protein. Hence, ohanin together with its pro-protein domain was named pro-ohanin. From the deduced amino acid sequence analysis, the cleavage of pro-ohanin to produce the mature ohanin appears to occur at the dibasic RR site.

### Sequence Alignment of Pro-Ohanin

Comparison of the full length cDNA sequence using the BLASTN algorithm (http://www.ncbi.nlm.nih.gov/BLAST/) (15) did not display any sequence homology with other nucleotide sequences deposited in the GenBank database so far. However, the deduced amino acid sequence showed homology to the complete PRY-SPRY and the B30.2-like domains with the presence of the 3^{rd} motif (LDYE) as proposed by Henry et al. (20, 21) at the pro-protein domain. An alignment of the deduced protein sequence with those proteins containing the B30.2-like domains is shown in FIG. 13. The protein sequence shared an overall identity of 38% and similarity of 49% to PRY-SPRY domains. Schematic representation of proteins possessing B30.2-like domain is shown in FIG. 14.

### Expression of Pro-Ohanin

Pro-ohanin was cloned into expression vector for expression using E. coli. Primers 19K1 and 19K2 were used to amplify, as well as add Met and stop codon, and restriction sites for cloning into vectorM. The amplified sequence, flanked by restriction sites, was digested and ligated into the expression vector at the BamHI site and NotI site. Usage of specific restriction sites for ligation mainly to ensure that the pro-ohanin was inserted in the proper orientation, whereas Met was inserted as the second alternative cleavage site Agarose gel showing the amplified fragment is shown in FIG. 15A. The schematic diagram of pro-ohanin expression vector construct is shown in FIG. 15B.

The final construct was transfected into E. coli strain DH5α and cloned. Plasmids from a positive clone was transfected into E. coli strain BL21(DE3) for the expression of recombinant pro-ohanin. A single colony was inoculated and grown as 100 ml seed cultures. The seed culture was further inoculated into a fresh LB medium at 1:50 dilution. The expression of pro-ohanin in bacterial culture was induced in the late logarithmic phase using 0.1 mM IPTG and analyzed by SDS-PAGE. Total protein prepared from bacterial culture after IPTG induction showed an increased expression of a band with an approximate molecular mass of 20 kDa (expected size of the fusion protein), when compared to that of the total protein from the uninduced culture (FIG. 16A).

The soluble fusion protein was purified from the total protein using a Ni-NTA column under non-denaturing conditions (FIG. 16B). The total yield of the fusion protein was approximately 50 mg/l bacterial culture. The purified fusion protein was subjected to thrombin cleavage (FIG. 16C). The yield obtained from thrombin cleavage was relatively higher than that obtained from CNBr cleavage. Recombinant pro-ohanin was purified using RP-HPLC (data not shown). RP-HPLC profile showed two distinctly separated peaks corresponding to the fusion peptide and the pro-ohanin, respectively. ESI-MS was used to determine the precise molecular mass and the homogeneity of pro-ohanin. Biospec Reconstruct spectra indicated that pro-ohanin was homogenous with a molecular mass of 19277.27±2.32 (FIG. 17). The N-terminal sequence of the first eight residues of pro-ohanin was GSMSPPGN as determined by Edman degradation sequencing. This sequence matched the predicted N-terminal sequence of pro-ohanin with Gly, Ser and Met as extra residues, left over from the thrombin cleavage.

### Secondary Structures of Recombinant Pro-Ohanin

Secondary structural contents of both ohanin and pro-ohanin were measured at the concentration of 12.5 μM by CD spectroscopy (FIG. 18A). The CD spectrum of ohanin shows a negative ellipticity extrema near 215 nm, indicating the presence of β-sheet structures (7% α-helix, 48% β-sheet, 45% random coil) (FIG. 18B). Unlike ohanin, the CD spectrum of pro-ohanin shows a mixture of secondary structural profile consisting of 22% α-helix, 29% β-sheet and 49% random coil. Hence, it is clear that the presence of C-terminal propeptide segment in pro-ohanin increases its α-helical contents as observed in FIG. 18. Both ohanin and pro-ohanin adopt ˜50% random coil structures.

### Functional Characterization of Pro-Ohanin

The purified pro-ohanin was investigated for its in vivo toxicity in mice. It was non-lethal up to the dose of 10 mg/kg when given i.p. There were no obvious signs of sluggishness as compared to those observed when mature ohanin was injected. In addition, no detectable hemorrhage or necrosis were found in the brain, heart, lungs, kidneys, spleen and liver by visual inspection when the mice were sacrificed after 24 h (data not shown).

### Effect of Pro-Ohanin on Locomotor Activity

As described in the previous chapter, ohanin induces a dose-dependant and statistically significant hypolocomotion after i.p. injection (F_{3,30}=5.787, p<0.01) (34). The effect of pro-ohanin on the locomotor activity of the mice was examined via i.p. injection at the doses of 0.1 mg/kg, 1 mg/kg and 10 mg/kg. The total movement counts of pro-ohanin at the highest dose (10 mg/kg dose) were approximately 2048±225 which was comparable to that from the controls (1942±147). Thus it is clear that pro-ohanin does not cause any significant inhibition on the mobility of the experimental mice after i.p. injection (F_{3,28}=0.251, p>0.05) (FIG. 19B) as compared to ohanin (FIG. 19A).

Pro-ohanin was also used to assess its direct effect on locomotion upon i.c.v. injection. Like wise, the doses used for i.c.v. injection were approximately 1000-fold lower than the doses used for i.p. injection. Interestingly, even at low dose (0.3 μg/kg; p=0.000), the total movement counts of the mice decreased to 190±43 as compared with the control mice (2109±264). Hence pro-ohanin exhibited high potency in inducing hypolocomotion in mice after i.c.v. injection for all the doses of 0.3 μg/kg, 1 μg/kg and 10 μg/kg (F_{3,25}=35.565, p=0.000) (FIGS. 19D and 19C).

### Effect of Pro-Ohanin on Hyperalgesia

Similar to ohanin, the effect of pro-ohanin on hot plate experiment was assessed 15 min after the injections. Pro-ohanin, when given intraperitoneally, shoes neither dose dependent nor U-shaped dose response curve as compared to that of the ohanin-injected mice (F_{3,28}=0.922, p>0.05) (FIGS. 20B and 20A). When injected intracerebroventricularly, pro-ohanin produces relatively short latency time for all the doses used (0.3 μg/kg, 1 μg/kg and 10 μg/kg) as compared to its controls (F_{3,39}=3.275, p<0.05) (FIG. 20D). The hyperalgesic effect was significant only at doses of 0.3 μg/kg and controls (p=0.026).

The observations from locomotion experiment and hot plate assay indicate an interesting differences in the pharmacological actions of ohanin and pro-ohanin. Ohanin induces hypolocomotion and hyperalgesia through both the i.p. and i.c.v. injection routes, whereas pro-ohanin is only active when given intracerebroventricularly.

### Southern Blot Hybridization

As mentioned above, there are two mRNA subtypes for ohanin. It was of interest to determine whether these two mRNAs are products of two independent genes or derived through alternative splicing. As a first step, we performed the genomic Southern hybridization experiment. King cobra genomic DNA was digested with EcoRI, HindIII, BamHI and NdeI, separately. These genomic DNA digests were hybridized with a 297-bp DIG-labeled probe designed from nucleotide 319 to 616 of its cDNA). We observed a single band in all four digests (FIG. 21), suggesting that ohanin is encoded by a single gene in the king cobra genome.

### Cloning and Sequencing of Ohanin Gene

To determine the genomic organization of ohanin gene, genomic DNA PCR and ‘genome walking’ approaches were used. Ohanin cDNA sequence was used to map the exon-intron boundaries. In the first amplification, gDNAsigpep and gDNAstop were used to amplify its coding region. The resultant fragment was ˜1.9 kb (data not shown). Our attempts to PCR amplify the 3′-UTR region of the genomic DNA yielded another ˜750 bp band.

We tried to amplify the 5′-UTR region from the genomic DNA using primers designed from the transcription start site to the signal peptide region. However, no band was obtained after several attempts. This led us to suspect that our primers may have been interrupted by the presence of intron(s) or the thermal cycling profile used was still not optimal. Hence, genome walker libraries were constructed. The ‘genome walk’ was performed using antisense primers, gDNA5UTR1 and gDNA5UTRnes2 with adaptor primers (AP1 and AP2) from the kit. The resultant ˜1.65 kb fragment was fully sequenced.

We obtained another ˜1.8 kb further upstream by a gDNA PCR performed using primers 1-gDNA5UTR and 2-gDNA5UTR designed from the 5′-region of the cDNA and the previously obtained ˜1.65 kb genomic DNA fragment (data not shown). With the optimized thermal cycling profile, we further generated another fragment of ˜1.4 kb corresponding to the transcription start site region of the cDNA using primers 9-gDNA5UTR and 6-gDNA5U TR (data not shown).

Thus, we have obtained a total of 7086 bp of the gene sequence, spanning from 5′-UTR to 3′-UTR regions of ohanin cDNA (FIGS. 22 and 23). Sequences flanking the splice junctions were determined for all the exons and introns of ohanin (FIG. 23A). The donor and acceptor splice sites of the exon-intron boundaries conform with the rule that intron begins with GT and ends with AG. Ohanin gene contains five exons and four introns. Out of five exons identified, the coding region of ohanin is made up from two exons. Exons 1, 2 and 3 encode mainly the 5′-UTR region. Interestingly, exon 2 is spliced out in one of the mRNA subtypes (FIGS. 1C, 4B and 4C). Exon 4 comprises of the remaining 5′-UTR region (11 bp), signal peptide and the first eight amino acid residues of ohanin. Exon 5 encodes for ohanin spanning from residues 9 to 107, the propeptide segment as well as the sequence corresponding to the 3′-UTR (FIGS. 22 and 23).

### In Vitro Binding Study of His-Pro-Ohanin

A previous study on the locomotor activity of mice showed that both ohanin and pro-ohanin exhibit potent hypolocomotion effect upon i.c.v. injection. Thus pro-ohanin with the His-tag fused at the N-terminal (named His-pro-ohanin) was first tested for its ability to bind on brain slices in vitro (FIG. 24). Our results show that His-pro-ohanin binds specifically to the hippocampus and cerebellum regions of the brain as shown in FIG. 25. The other regions of the brain slice remain unstained. Similarly, control experiments without pre-incubation with His-pro-ohanin showed no fluorescence staining (FIG. 25).

To further confirm its specificity, a competition binding control assay was performed (FIG. 26). The brain slice was pre-incubated with native ohanin, followed by His-pro-ohanin before staining. Fluorescence stain was seen to be reduced (FIG. 27). Thus our results indicate His-pro-ohanin binds specifically to the brain and this interaction appears to be mediated through the region in the mature protein.

### In Vivo Binding Study of His-Ohanin and His-Pro-Ohanin

Next, we sought to confirm whether ohanin and/or pro-ohanin crosses the blood-brain barrier to exert the observed pharmacological actions (FIG. 28). Thus His-ohanin and His-pro-ohanin were injected via i.p. and i.c.v. routes, and the presence of the proteins in the brains, at three different concentrations, were detected using immunofluorescence. FIG. 29 shows that His-ohanin binds specifically to the cerebellum and hippocampus in a dose-dependant manner in vivo. However, when His-pro-ohanin was administered, a markedly reduced staining was observed in the same regions (FIG. 30). Although this shows that pro-ohanin could pass the blood brain barrier, it should be noted that the amount of protein administered was 1000-fold higher than the highest previously reported levels (10 mg/kg), to allow proper immunofluorescence staining.

Results obtained supports our previous report that ohanin exhibits its effects by affecting the central nervous system directly, and that ohanin is able pass the blood-brain barrier and transverse into the cranial space. In addition, it also suggests that the precursor protein, pro-ohanin, cannot transverse as efficiently, if at all (FIG. 30).

### Discussion

We identified the presence of a novel protein with an unusual molecular mass using initial screening of king cobra venom by LC-MS (FIG. 1). Here we describe the purification and characterization of this protein, ohanin. The complete amino acid sequence of ohanin was determined by Edman degradation. It has 107 amino acid residues with a single cysteine residue (FIG. 3). It does not have similarity to any of the well established families of snake venom proteins. Thus ohanin and Thai cobrin (isoform reported from Thai cobra) are the first members of a new family of snake venom proteins. The unique feature of the members of this family appears to be the low content of cysteine residues (<1%). In contrast, the members of all the other snake venom protein families have multiple disulfide bonds and high contents of cysteine residues (generally more than 8 to 10%).

### Ohanin and B30.2-Like Domain Proteins

CDD search revealed that ohanin shares similarity with the PRY-SPRY domains (FIG. 5). Three copies of SPRY domains were first identified in three mammalian ryanodine receptor (RyR) subtypes. This domain is also present in three copies in a dual-specificity kinase, splA, found in Dictyostelium discoideum. Owing to the repeats in splA and RyR, these sequences are therefore referred to as SPRY domain (23). The SPRY domain has been identified as a sub-domain within the B30.2-like domain family (19). The SPRY domain, when compared to the B30.2-like domain, has a deletion at the N-terminal region. It is interesting to note that the PRY domain, which comprises of ˜50 residues, has always been found at the N-terminal region of SPRY domain (˜110 to 120 residues). Hence, both PRY-SPRY domains could be regarded as sub-domains of the B30.2-like domains.

The B30.2 domain is a conserved protein domain of around 160 to 170 amino acids which is encoded by a single exon, mapping within the Human Class I Histocompatibility Complex (MHC) region (24). It was, therefore, named after the B30.2 exon in the MHC I region in which it was originally identified. The B30.2-like domain occurs in nuclear, cytoplasmic, transmembrane or secreted proteins, particularly at the C-terminal regions and these proteins are classified according to the type and/or the function of N-terminal domains (20, 21). The first category comprises a subset of RING (Really Interesting New Gene) finger proteins with BBox and coiled-coil domain. The second category comprises of BTN (butyrophilin) and the BTN2/BTN3 putative proteins with two immunoglobulin-like folds of variable (IgV) and constant 1 (IgC1) types. The third category comprises of stonustoxin, a lethal toxin isolated from venom of stonefish Synanceja horrida. In addition, enterophilins, SOCS box (suppressor of cytokine signaling) and vitamin-K-dependent gamma carboxylases families also contain the B30.2-like domain at their C-terminal regions. Although the B30.2-like domain proteins are found in diverse species and in different protein contexts, the function(s) of the B30.2-like domain is not clearly understood yet (20, 21). Based on the structural similarity, we include ohanin as a new member of the rapidly expanding B30.2-like domain family. However, it appears to be unrelated to any of the other classes of proteins containing the B30.2-like domains. It is interesting to note that ohanin has a relatively short N-terminal region of only 8 amino acid residues as compared to that of other proteins containing B30.2-like domains. In addition, ohanin also has a shorter C-terminal region, lacking the 50 to 60 amino acid residues at the C-terminal region of the B30.2-like domain. Similar C-terminal truncation in the B30.2-like domain is also found in KIAA0129 isolated from human cell line KG-1 (gb: D50919) and Staf50 (gb: X82200) (FIG. 5).

### Biological Function(s) of Ohanin

Ohanin induces hypolocomotion in experimental mice by i.p. injection in a dose-dependent manner (FIG. 4). It should be noted that neurotoxins in snake venoms are particularly important in inducing paralysis of skeletal muscles (25). To test whether ohanin induces blocking of peripheral neuromuscular junction, we studied its effect on isolated chick biventer cervicis nerve-muscle preparations (CBCNM). Ohanin possesses no effect on the direct twitch response of the CBCNM stimulation as well as on the responses to exogenously applied agonists, such as ACh, CCh and KCl (Y. F. Pung, J. C. Wickramaratna, N. G. Lumsden, W. C. Hodgson, and R. M. Kini, unpublished observations). These results indicate that ohanin is devoid of both presynaptic and postsynaptic toxicity (including myotoxicity). Therefore, we directly injected ohanin into the ventricles of the mice to examine its pharmacological actions in the central nervous system. Ohanin produced ˜6,500-times more potent hypolocomotion activities when injected i.c.v. compared to i.p. injections. Thus, ohanin induces hypolocomotion that is presumably mediated by a direct action on the central nervous system. Further studies are underway to determine whether ohanin crosses the blood-brain barrier.

In hot plate assay, both the i.p. and i.c.v. injection routes induced a similar U-shaped dose-response curve (FIGS. 7A and 7B). Although lower and intermediate doses showed shorter average latency times, there were no obvious differences in the latency time between the high doses and the respective controls. The results suggest that the effects of locomotor impairment caused by ohanin need to be considered when interpreting the results from hot plate assay which is dependent on a normal functioning motor system. The increase in the latency time at higher doses of ohanin administered may have been caused by severe impairment in the movements. Therefore, the mice would not be able to respond immediately to the thermal pain experienced. Again, the ability of the protein to elicit a response at greatly reduced doses for i.c.v. injection as compared to systemic administration in the hot plate assay strongly suggests that ohanin probably has a direct effect at the central nervous system. However, the exact mode of action of ohanin is yet to be investigated.

Butyrophilin is involved in the budding and release of milk-fat globules during lactation (24, 26). Its B30.2-like domain interacts with xanthine dehydrogenase/oxidase and this interaction appears to be important for its function (26, 27). Based on the assumption that proteins containing similar domains exert their functions through similar protein-protein interaction and mechanisms, Henry et al. (20, 21) proposed a mechanism for the hypotensive action of SNTX that is mediated through the release of endothelium-derived relaxing factor (probably NO or NO-yielding substances). Accordingly, SNTX through its B30.2-like domain would interact with xanthine oxidase relieving the xanthine oxidase-mediated inhibition of NO synthase. This in turn would lead to increased synthesis of NO and vasorelaxation (20, 21, 28, 29). In our study, ohanin did not exhibit any significant effect on the blood pressure in anaesthetized Sprague-Dawley rats up to the dose of 1 mg/kg when given intravenously (Y. F. Pung, S. M. Atan, S. Moochhala, and R. M. Kini, unpublished observations). Although we have not examined the direct interaction of ohanin with xanthine oxidase, we propose that ohanin's function is independent of xanthine oxidase.

### Design of the Synthetic Gene and Cloning of Ohanin

We are interested in the study of structure-function relationships of small and novel venom proteins from snakes (7). For these studies, which lead to the use of venom proteins as models for drug design and anti-venoms, a reliable and inexpensive method for obtaining the proteins is needed. One potential method for obtaining the proteins is to produce them using solid-phase peptide synthesis and combinatorial chemistry. A second, less expensive method is to over-express the protein in bacterial hosts using molecular biology techniques. Synthetic gene for the production of proteins is a powerful approach. In this approach, either single amino acid or entire protein domain changes can be easily achieved as compared to cDNA sequence (30).

The synthetic gene was designed as follows: first, the amino acid sequence of ohanin was reverse-translated into nucleotide sequence through the use of the triplet codons that occur most frequently in E. coli (31). Second, a total of six common restriction sites were added at the 5′- and 3′-region of the synthetic gene for easy sub-cloning into a wide range of expression vectors. Third, 10 unique restriction sites were introduced, without changing the encoded amino acid sequence, into the sequence for future cassette-based mutagenesis. The goal was to produce a nucleotide sequence which contained restriction sites that for a variety of restriction enzymes would cleave the gene only once. In addition, the restriction sites flanked conserved sequence motifs of B30.2-like domain and cysteine residue of the gene, and were present approximately every 20 to 45 bp. Such a construction would permit the easy manipulation of the encoded amino acid sequence by digestion with two restriction endonucleases, removal and ligation of the replacement DNA segment. Fourth, codons for Met-Ala residues were incorporated at the N-terminal of obanin to facilitate CNBr cleavage from the fusion protein after expression as ohanin does not contain any Met residue in its amino acid sequence. Fifth, a stop codon was introduced after the last amino acid residue to stop the translation process. Finally, the sequence was checked by various computer programs such as DNAman and DNAsis followed by visual inspection for undesired restriction sites and potential for excess secondary structures (FIG. 3). Using this synthetic gene, we produced the recombinant ohanin in E. coli. The recombinant ohanin resembles the native protein in its folding and function as determined by CD (FIG. 11) and biological activity (FIG. 7C). Thus the designed synthetic gene will be important for future study on the structure-function relationships of this novel protein.

### Physiological Role(s) of Ohanin

Snake venoms are complex mixture of pharmacologically active peptides and proteins. They play important role in both offensive and defensive functions. Some of these proteins, such as neurotoxins, are involved in paralyzing the prey, while others including hydrolytic enzymes may be involved in digesting the prey animals. We propose that ohanin could contribute by slowing down the mobility of the prey and help in its capture. The hyperalgesic effect may also help in the defensive function by inducing pain in predatory animals. Further studies are needed to clarify the role played by ohanin in relation to the other components present in the venom.

### Implications of the Propeptide Segment of Pro-Ohanin

Ohanin is synthesized as a prepro-protein in the venom glands with a C-terminal propeptide segment (FIG. 12). This is the first snake venom protein reported so far harbouring a propeptide segment at the C-terminal of the mature protein.

Proprotein was expressed and purified from E. coli for characterization. Recombinant pro-ohanin is obtained as highly soluble protein, in contrast to recombinant ohanin which is insoluble after the expression despite efforts made to increase its solubility. Hence it is clear that the presence of propeptide segment helps to solubilize the mature protein after the large scale expression in E. coli. However, it is not clear whether the propeptide segment aids to increase the solubility and/or the proper folding of the mature protein in the venom gland cells or lumen. It should be noted that mature protein is present in trace amount in the crude venom. Thus this may suggest that the propeptide segment may not be required for the solubility of ohanin in the venom.

Similar to ohanin, pro-ohanin was also assessed for its biological functions in mice. It should be noted that analyses from both the locomotor activity and hot plate assay strongly indicate that pro-ohanin did not exhibit similar pharmacological actions in intraperitoneally-administered mice as compared to the mature ohanin. But pro-ohanin shows potent hypolocomotion and hyperalgesia effects when injected directly into the mice ventricles. The large size and/or conformation changes of the proprotein may have inhibited pro-ohanin from crossing the blood-brain barrier and subsequently preventing its interaction with molecular target(s) at the central nervous system. Interestingly, although the presence of propeptide segment inhibits the ability of ohanin to cross the blood-brain barrier, it enhances the pharmacological effects of ohanin at the central nervous system. It should be noted that pro-ohanin is ˜35-fold more potent than ohanin when the injection is given via i.c.v. route. Furthermore, pro-ohanin at 0.3 μg/kg dose is able to block ˜90% of the locomotor activity of the experimental mice.

### Origin of Exons

We have also shown that ohanin gene has a single intron which is located just before the PRY-SPRY and B30.2 domains (FIGS. 22 and 23). There are two current views on the origin of the introns. One theory, exon early, states that exons are descendants of ancient minigenes and the introns represent the spacing between them (32). The other theory, introns-late, states that split genes arise from uninterrupted genes by the insertion of introns (33). In brief, the first theory suggests that exons represent discrete functional or structural units of protein, whereas the second theory suggests that the insertion of introns is somewhat random. The similarity in organization in pro-ohanin gene indicates that it has probably evolved from the same ancestral gene as B30.2 domain proteins. This further indicates that the evolution of ohanin gene was more in line with the exon early theory.

### Mechanism of Ohanin and Pro-Ohanin

Localization of ohanin and pro-ohanin to the hippocampus and cerebellum suggests the exact nature of the protein to exhibit the hypolocomotive effects. The hippocampus has been shown to affect the metabolism, which indirectly affects the locomotive ability of the animals. Indeed, neurons in the hippocampus were implicated in the hyperlocomotion caused by phenylcycliprin and cocaine. It could be possible that ohanin acts on the same receptors as an antagonist to show its hypolocomotive effects. On the other hand, cerebellum was implicated in the overall balance of the animal. Previous experiments using amphetamines show that the neurons in the cerebellum are affected, and show hyperlocomotion as a result of amphetamine administration. It could be also possible that the same neurons are also involved in ohanin interaction. It is yet unknown if the neurons which affect balance may be affected by ohanin, although the lack of balance might induce a lack of overall locomotive abilities.

### CONCLUSION

We have identified, purified and functionally characterized a novel protein, ohanin from king cobra venom. Ohanin induces hypolocomotion and hyperalgesia in mice. The effectiveness of ohanin administered by i.c.v. route as compared to systemic administration strongly suggests its action through the central nervous system although the role of peripheral nervous system cannot be ruled out. We have also established a synthetic gene expression system for its future structure and function relationship studies. The detailed mechanism of action(s) of ohanin at the molecular level is currently under investigation.

In addition, we have also cloned and sequenced the cDNA for ohanin. Its full-length cDNA sequence of 1558 bp encodes for prepro-ohanin with a propeptide segment at the C-terminal. Recombinant pro-ohanin shows potent effect on locomotion when injected i.c.v. but not when injected i.p. Thus maturation appears to be crucial for the biological activity of ohanin. The genomic DNA sequencing indicates the presence of five exons and four introns. Interestingly, the second exon encoding the 5′-untranslated region is alternatively spliced. All these findings together indicate that ohanin forms a new subfamily of B30.2 domain-containing proteins.

### REFERENCES

1. Harvey, A. L. (1991) Snake toxins, Pergamon Press, NY
2. Lee, C. Y. (1979) Snake venoms, Springer-Verlag, NY
3. Kordis, D., and Gubensek, F. (2000) Gene 261, 43-52
4. Dufton, M. J. (1993) Endeavor 17, 138-142
5. Menez, A (1998) Toxicon 36, 1557-1572
6. Kini, R. M. (2002) Clin. Exp. Pharmacol. Physiol. 29, 815-822
7. Torres, A. M., Wong, H. Y., Desai, M., Moochala, S., Kuchel, P. W., and Kini, R. M. (2003) J. Biol. Chem. 278, 40097-40104
8. Yamazak Y., Hyodo, F., and Morita, T. (2003) Arch. Biochem. Biophys. 412, 133-141
9. Mochca-Morales, J., Martin, B. M., and Possani, L. D. (1990) Toxicon 28, 299-309
10. Howard-Jones, N. (1985) WHO Chron. 39, 51-56
11. Joseph, J. S., Chung, M. C. M., Jeyaseelan, K, and Kini, R. M. (1999) Blood 94, 621-631
12. Inglis, A. S. (1983) Meth. Enzymol. 91, 324-332
13. Paxinos, G., and Franklin, K. B. J. (2004) The mouse brain in stereotaxic coordinates, 1^{st} Ed., Elsevier Science, USA
14. Woolfe, D., and MacDonald, A. D. (1944) J. Pharmacol. Exp. Ther. 80, 300-307
15. Laemmli, U. K. (1970) Nature 227, 680-685
16. Altschul, S. F., Madden T. L., Schaffer, A. A., Zhang, J. H., Zhang, Z., Miller, W, and Lipman, D. J. (1997) Nucleic Acids Res. 25, 3389-3402
17. Marchler-Bauer, A., Anderson, J. B., DeWeese-Scott, C, Fedorova, N. D., Geer, L. Y., He, S., Hurwitz, D. L, Jackson, J. D., Jacobs, A. R., Lanczycki, C. J., Liebert, C. A., Liu, C., Madej, T., Marchler, G. H., Mazumder, R., Nikolskaya, A. N., Panchenko, A. R., Rao, B. S., Shoemaker, B. A., Simonyan, V., Song, J. S., Thiessen, P. A., Vasudeyan, S., Wang, Y., Yamashita, R. A., Yin, J. J., and Bryant, S. H. (2003) Nucleic Acids Res. 31, 383-387
18. Doolittle, R. F. (1995) The multiplicity of domains in proteins. Annu. Rev. Biochem. 64, 287-314
19. Seto, M. H., Liu, H. L. C., Zajchowski, D. A., and Whitlow, K (1999) Proteins. 35, 235-249
20. Henry, J., Ribouchon, M. T., Offer, C., and Pontarotti, P. (1997) Biochem. Biophys. Res. Commun. 235, 162-165
21. Henry, J., Mather, I. H., Mcdermott, M. F., and Ponttarotti, P. (1998) Mol. Biol. Evol. 15, 1696-1705
22. Prodromou, C., and Pearl, L. H. (1992) Protein Eng. 5, 827-829
23. Ponting, C. P., and Bork, P. (1997) TIBS. 22, 193-194
24. Vernet, C., Boretto, J., Mattei, M., Takashi, M., Jack, L. J. W., Mather, I. H., Rouquier, S., and Pontarotti, P. (1993) J. Mol. Evol. 37, 600-612
25. Hodgson, W. C., and Wickramaratna, J. C. (2002) Clin. Exp. Pharmacol. Physiol. 29, 807-814
26. Ishii, T., Aoki, N, Noda, A., Adachi, T., Nakamura, R., and Matsuda, T. (1995) Biochim. Biophys. Acta 1245, 285-292
27. Banghart L. R., Chamberlain, C. W., Velarde, J., Korobko, I. V., Ogg, S. L., Jack, L. J. W., Vakharia, V. N., and Mather, I. H. (1998) J. Biol. Chem. 273, 4171-4179
28. Low, K. S. Y., Gwee, M. C. E, Yuen, R, Gopalakrishnakone, P., and Khoo, H. E. (1993) Toxicon 31, 1471-1478
29. Sung, J. M. L., Low, K. S. Y., and Khoo, H. E (2002) Biochem. Pharmacol. 63, 1113-1118
30; Jones, H. M.; Kubo, A., and Stephens, R. S. (2000) Gene 258, 173-181
31. Sharp, P. M., and Li, W. H. (1987) Nucleic Acids Res. 15, 1281-1295
32. Gilbert, W., and Glynias, M. (1993) Gene 135, 137-144
33. Stoltzfus, A, Spencer, D. F., Zuker, M., Logsdon, J. M., and Doolittle, W. F. (1994) Science 265, 202-207
34. Pung Y. F., Kumar S. V., Rajagopalan N., Kumar P. P., Fry B. G., and Kini R. M. (2005)

1. A protein, selected from the group consisting of:
(a) a protein comprising the sequence SEQ ID NO: 1;
(b) a protein comprising the sequence SEQ ID NO: 3;
(c) a protein comprising the sequence SEQ ID NO: 5;
(d) a protein comprising the sequence SEQ ID NO: 7; or
(e) (i) an allelic variant of a protein according to (a), (b), (c) or (d);
(ii) a functional equivalent of a protein according to (a), (b), (c), (d) or (e)(i) which functional equivalent retains the ability to induce at least one of hypolocomotion or hyperalgesia, or which functional equivalent has an antigenic determinant in common with a protein according to (a) s (b), (c), (d) or (e)(i);
(iii) an active fragment of a protein according to (a), (b), (c), (d), (e)(i) or (e)(ii) which active fragment retains the ability to induce at least one of hypolocomotion or hyperalgesia, or which active fragment has an antigenic determinant in common with a protein according to (a), (b), (c), (d), (e)(i) or (e)(ii); or
(iv) a fusion protein comprising a protein according to (a), (b), (c), (d), (e)(i), (e)(ii) or (e)(iii).

2. A nucleic acid molecule which encodes the protein according to the claim 1.

3. A vector that contains the nucleic acid molecule according to claim 2.

4. A host cell transformed with the vector according to claim 3.

5. A method of producing the protein according to claim 1, the method comprising culturing a host cell according to claim 4.

6. A method of producing the protein according to claim 1, the method comprising performing chemical synthesis of the protein.

7. The method according to claim 6, wherein said chemical synthesis is a solid phase peptide synthesis or combinatorial chemistry.

8. A method of making a polyclonal or monoclonal antibody, capable of binding the protein according to claim 1, wherein the method comprises immunizing an animal with a protein according to claim 1 and harvesting antibodies from the animal or harvesting cells from the animal for use in producing monoclonal antibodies.

9. An antibody which binds to the protein according to claim 1.

10. A method of producing an antivenom against a protein according to claim 1 wherein the method comprises immunizing an animal with a protein according to claim 1 and harvesting antibodies from the animal for use as the antivenom.

11. The method according to claim 10 wherein the animal is a horse, goat, sheep or bird.

12. An antivenom effective against a protein of claim 1.

13. (canceled)

14. A pharmaceutical composition comprising the protein according to claim 1, the nucleic acid molecule according to claim 2, the vector according to claim 3, the host cell according to claim 4, the antibody according to claim 9, or the antivenom produced by the method according to claim 11.

15. (canceled)

16. (canceled)

17. (canceled)

18. A method of sedating an animal comprising administering the protein according to claim 1, the nucleic acid molecule according to claim 2, the vector according to claim 3, or the host cell according to claim 4.

19. A method of treating a patient with a neurological or muscular disease comprising administering to the patient the protein according to claim 1, the nucleic acid molecule according to claim 2, the vector according to claim 3, or the host cell according to claim 4.

20. (canceled)
